(19) United States
(12) Patent Application Publication
(10) Pub. No.: US 2024/0117001 A1
(43) Pub. Date: Apr. 11, 2024

(54) METHOD OF IDENTIFYING MALODOR MODULATING COMPOUNDS

(71) Applicant: FIRMENICH SA, Satigny (CH)

(72) Inventors: Patrick Pfister, Plainsboro, NJ (US); Matthew Rogers, Plainsboro, NJ (US); Huey-Ling Kao, Plainsboro, NJ (US); Claude Ayome Abibi, Plainsboro, NJ (US)

(73) Assignee: FIRMENICH SA, Satigny (CH)

(21) Appl. No.: 18/486,417

(22) Filed: Oct. 13, 2023

Related U.S. Application Data
(63) Continuation of application No. 16/492,115, filed on Sep. 7, 2019, now Pat. No. 11,787,847.
(60) Provisional application No. 62/469,256, filed on Mar. 9, 2017.

(51) Int. Cl. C07K 14/705; G01N 33/50

(52) U.S. Cl. CPC C07K 14/705; G01N 33/5008; G01N 2500/20

(57) ABSTRACT

Provided herein are polypeptides that bind to the malodour-causing substance DMTS. Also provided are nucleic acid sequences that encode for the polypeptides. Further provided herein is a method for identifying a compound that binds, suppresses, blocks, inhibits, and/or modulates the activity of one or more olfactory receptor that is activated by the malodor-causing substance DMTS comprising a) contacting the receptor, or a chimera or fragment thereof with a compound and b) determining whether the compound has an effect on the activity of the receptor. Further provided is an expression vector comprising the nucleic acid encoding the polypeptides described as well as a non-human organism or a host cell modified to express a receptor that is activated by DMTS. Also provided is the use of the polypeptides for identifying malodor modulating compounds.

### CROSS-REFERENCE TO RELATED APPLICATIONS

The present application is a continuation of U.S. application Ser. No. 16/492,115, filed Sep. 7, 2019, which is a U.S. national phase entry under 35 U.S.C. § 371 of International Application No. PCT/EP2018/055753, filed Mar. 8, 2018, which claims priority to U.S. Provisional Patent Application No. 62/469,256, filed on Mar. 9, 2017. The entire contents of these applications are explicitly incorporated herein by this reference.

### SEQUENCE LISTING

This application contains a Sequence Listing which has been submitted in XML file format with this application, the entire content of which is incorporated by reference herein in its entirety. The Sequence Listing XML file submitted with this application is entitled “10020_st_26.xml”, was created on Aug. 24, 2023 and is 80,026 bytes in size.

### FIELD

The technical field is directed to odorant and aroma receptors and assays that can be used to identify odorant and/or aroma compounds more specifically inhibitors, modulators or counteractants of malodor compounds such as dimethyl trisulfide (DMTS).

### BACKGROUND

Olfaction is one of the most complex and poorly understood of human sensory systems. From olfactory receptor (OR) activation to perception, there are many steps that still require further investigation. If we can understand how the OR code for individual odorants and mixtures translates into perception then we can exploit this knowledge to bring significant benefit in several areas. These areas include odor modulators like malodor counteractants that block the perception of unpleasant odors, new flavor and fragrance ingredients that replace non-biodegradable or toxic compounds, and odor enhancers that would limit our reliance on difficult to source compounds from natural sources. The ‘olfactory code’ combinatorial paradigm is centered on the observation that any single OR may be activated by multiple odorants, and conversely most odorants are capable of activating several ORs. In the mouse genome there are 1,200 distinct intact ORs. Humans, by contrast, have ˜400. In both cases, the repertoire of ORs is activated by many thousands of odorants in the world, and it is this combinatorial complexity that allows for the breadth of olfactory sensations we can perceive. However, odorants or ligands for only 82 mouse (˜8%) and 17 human ORs (˜10%) have been identified as of 2014 using traditional deorphanization methods. In addition, the physiological relevance of most ligands for the human ORs, essentially identified in vitro, has not been tested.

A method that can rapidly and reliably identify a relatively small subset of ORs, within the entire repertoire of ORs that exist in an organism that are specifically activated or inhibited by one or more odorants is described in WO2014/210585. However, using this method, there remains a need to identify odorant receptors and more particularly malodor receptors that are activated by particular malodor-causing substances.

Malodor-causing compounds such as dimethyl trisulfide (DMTS) and other closely related polysulfide compounds such as dimethyl disulfide (DMDS) can generate unpleasant odors that arise, for example, from latrines and other “bathroom” sources that contain fecal matter or from bad breath. Hence, malodor modulators or counteractants that bind, suppress, block, inhibit, and/or modulate the activity of one or more olfactory receptor that is activated by a particular malodor-causing substance such as DMTS or DMDS are desirable. Assays that rely on such new malodor receptors or on malodor receptors newly identified as associated with particular malodors-causing substances to identify new compounds or compounds mixtures that bind to these receptors are further desired.

### SUMMARY

Provided herein is a non-human host organism or a host cell that has been modified to express a receptor that is activated by DMTS wherein the receptor is selected from the group consisting of Olfr1193, Olfr1093, Olfr1097, Olfr166, Olfr169, Olfr738, Olfr742, Olfr207, Olfr665, Olfr669, Olfr1211, OR52N5, OR2L13, OR2AJ1, OR4C15, OR5AC2 OR8H3 OR11G2, OR52N2, and OR5T1.

Further provided is a non-human host organism or a host cell transformed to express a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40.

Further provided herein is an expression vector comprising a nucleic acid that encodes a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40.

Also provided herein is an expression vector having a nucleic acid wherein the nucleic acid comprises a nucleotide sequence having at least 70%, 75%, 80%, 85%, 90%, 95%, 96%, 97%, 98%, 99%, or 100% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 39, SEQ ID NO: 31, SEQ ID NO: 33, SEQ ID NO: 35, SEQ ID NO: 37, SEQ ID NO: 39, or the reverse complement thereof.

Provided herein is a nucleic acid comprising a nucleic acid sequence as recited above, and also provided is a polypeptide comprising an amino acid sequence as recited above.

Further provided herein is a method for identifying a compound that binds, suppresses, blocks, inhibits, and/or modulates the activity of at least one olfactory receptor that is activated by a malodor-causing substance such as dimethyl trisulfide (DMTS) or dimethyl disulfide (DMDS), wherein the receptor is a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40, wherein the method comprises:
a) contacting the receptor, or a chimera or fragment thereof with a compound;
b) determining whether the compound has an effect on the activity of the receptor.

Also provided is a method for identifying a compound that binds, suppresses, blocks, inhibits, and/or modulates the activity of at least one olfactory receptor that is activated by a malodor-causing substance comprising:
a. contacting a test substance and a malodor-causing substance to at least one olfactory receptor selected from the group consisting of Olfr1193, Olfr1093, Olfr1097, Olfr166, Olfr169, Olfr738, Olfr742, Olfr207, Olfr665, Olfr669, Olfr1211 OR52N5, OR2L13 OR2AJ1, OR4C15 OR5AC2 OR8H3 OR11G2, OR52N2, and OR5T1;
b. measuring the response of the olfactory receptor to the malodor-causing substance by measuring the response of the olfactory receptor in the presence and absence of the test substance;
c. identifying a test substance that modulates the response of the olfactory receptor on the basis of the response that was measured in the presence and absence of the test substance; and
d. selecting the identified test substance as a compound that modulates the response of the olfactory receptor to the malodor-causing substance, wherein the malodor-causing substance is dimethyl trisulfide (DMTS) or dimethyl disulfide (DMDS).

Further provided is a method for identifying a malodor modulator comprising:
a. contacting a test substance and a malodor-causing substance to at least one olfactory receptor, wherein the receptor comprises a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40;
b. measuring the response of the olfactory receptor polypeptide to the malodor-causing substance;
c. identifying, based on the measured response, a test substance that can suppress the response of the olfactory receptor; and
d. selecting, as a malodor modulator, the test substance that that binds, suppresses, blocks, inhibits, and/or modulates the response of the olfactory receptor,
wherein the malodor-causing substance is dimethyl trisulfide (DMTS) or dimethyl disulfide (DMDS).

Additionally provided is a recombinant nucleic acid molecule comprising: a nucleic acid comprising at least one of a Lucy tag, a FLAG® tag, and/or a Rho tag and a nucleic acid encoding a receptor selected from the group consisting of Olfr1193, Olfr1093, Olfr1097, Olfr166, Olfr169, Olfr738, Olfr742, Olfr207, Olfr665, Olfr669, Olfr1211, OR4S2, OR52N5, OR2L13, OR2AJ1, OR4C15, OR5AC2, OR8H3, OR11G2, OR52N2, and OR5T1 or the reverse complement thereof.

Still yet further provided is a non-human host organism or a host cell that is recombinantly modified to express a nucleic acid or a polypeptide as described above.

### DETAILED DESCRIPTION OF THE DRAWINGS

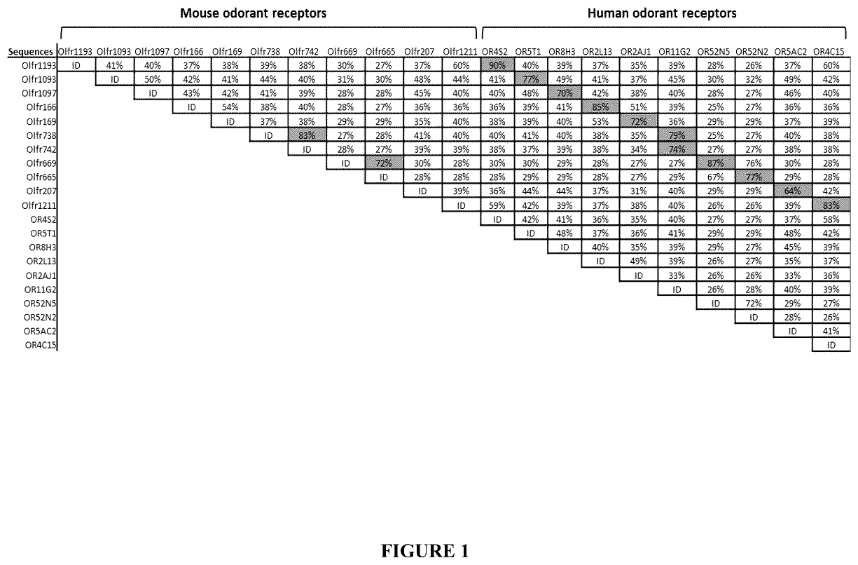

FIG. 1 shows the pairwise amino acid identity levels between the receptors mentioned herein.

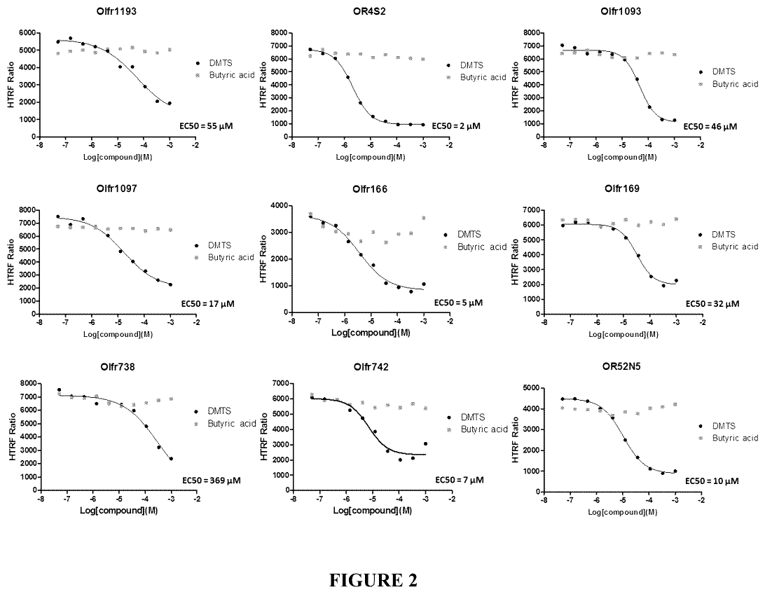

FIG. 2 shows DMTS dose response curves of mouse odorant receptors Olfr1193, Olfr1093, Olfr1097, Olfr166, Olfr169, Olfr738 and Olfr742, and of human odorant receptors OR4S2 and OR52N5.

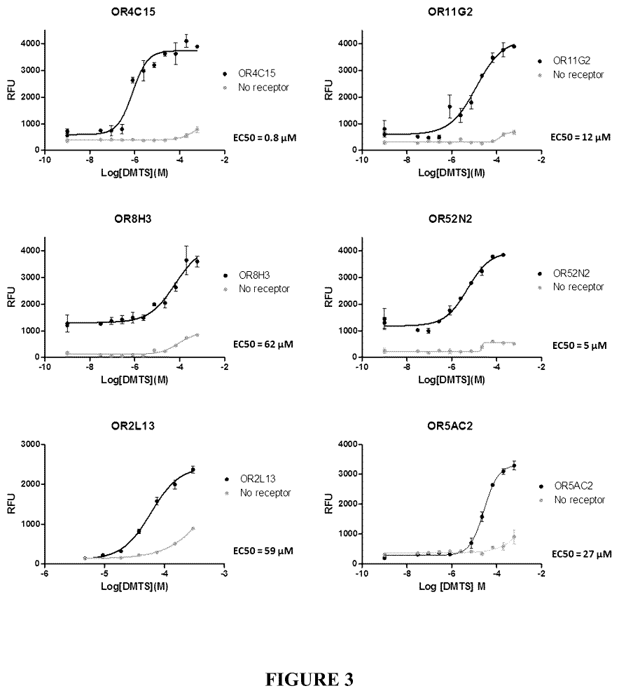

FIG. 3 shows DMTS dose response curves of human odorant receptors OR2L13, OR4C15 OR8H3 OR11G2 and OR52N2.

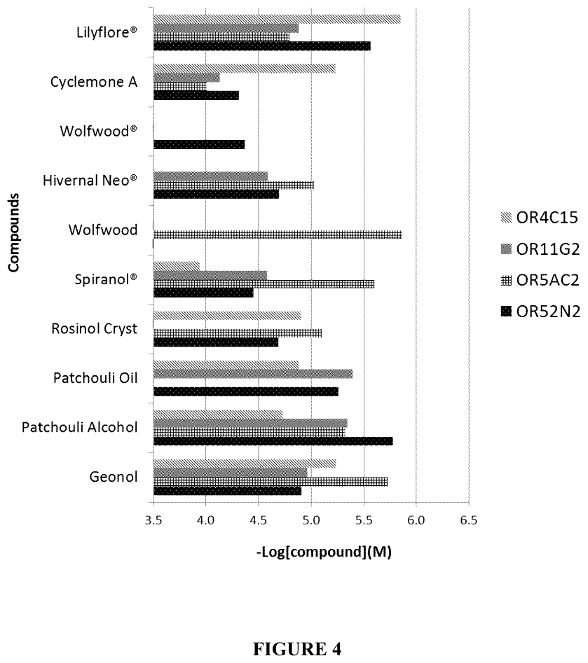

FIG. 4 shows a ranking of volatile compounds with specific inhibitory effects on DMTS receptor activity.

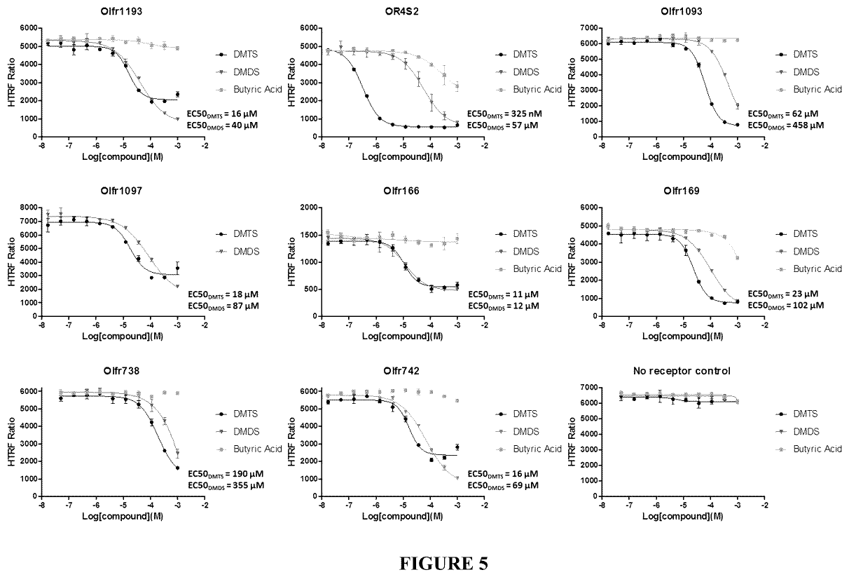

FIG. 5 shows DMTS and DMDS dose response curves of mouse odorant receptors Olfr1193, Olfr1093, Olfr1097, Olfr166, Olfr169, Olfr738, Olfr742 and of human odorant receptor OR4S2.

### DETAILED DESCRIPTION

For the descriptions herein and the appended claims, the use of “or” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The following terms have the meanings ascribed to them unless specified otherwise.

“OR” refers to one or more members of a family of G protein-coupled receptors (GPCRs) that are expressed in olfactory cells. Olfactory receptor cells can also be identified on the basis of morphology or by the expression of proteins specifically expressed in olfactory cells. OR family members may have the ability to act as receptors for olfactory signal transduction.

“DMTS OR” or “DMDS OR” refers to a member of the family of G protein-coupled receptors that is expressed in an olfactory cell, which receptors bind and/or are activated by DMTS or DMDS in a binding or activity assay for identifying ligands that bind and/or activate GPCRs. Such assays are described below. DMTS or DMDS receptors herein will include fragments, variants, including synthetic and naturally occurring, and chimeras or recombinant nucleic acids or proteins that respond to or bind DMTS or DMDS.

“OR” polypeptides are considered as such if they pertain to the 7-transmembrane-domain G protein-coupled receptor superfamily encoded by a single ˜1 kb long exon and exhibit characteristic olfactory receptor-specific amino acid motifs. The seven domains are called “transmembrane” or “TM” domains TM I to TM VII connected by three “internal cellular loop” or “IC” domains IC I to IC III, and three “external cellular loop” or “EC” domains EC I to EC III. The motifs and the variants thereof are defined as, but not restricted to, the MAYDRYVAIC motif (SEQ ID NO: 53) overlapping TM III and IC II, the FSTCSSH motif (SEQ ID NO: 54) overlapping IC III and TM VI, the PMLNPFIY motif (SEQ ID NO: 55) in TM VII as well as three conserved C residues in EC II, and the presence of highly conserved GN residues in TM I [Zhang, X. & Firestein, S. Nat. Neurosci. 5, 124-133 (2002); Malnic, B., et al. Proc. Natl. Acad. Sci. U.S.A 101, 2584-2589 (2004)].

“OR” nucleic acids encode a family of GPCRs with seven transmembrane regions that have “G protein-coupled receptor activity,” e.g., they may bind to G proteins in response to extracellular stimuli and promote production of second messengers such as IP3, cAMP, cGMP, and Ca′ via stimulation of enzymes such as phospholipase C and adenylate cyclase.

“Paralogous” OR genes or “paralogs” are the result of gene duplications and refer to closely related homologous genes within the same species. “Orthologous” OR genes or “orthologs” are defined as phylogenetically linked by a gene present in a common ancestor and refer to closely related homologous genes in other species.

The “N terminal domain” region starts at the N-terminus and extends to a region close to the start of the first transmembrane region. “Transmembrane regions” comprise the seven “transmembrane domains,” which refers to the domain of OR polypeptides that lies within the plasma membrane, and may also include the corresponding cytoplasmic (intracellular) and extracellular loops. The seven transmembrane regions and extracellular and cytoplasmic loops can be identified using standard methods such as hydrophobicity profiles, or as described in Kyte & Doolittle, J. Mol. Biol., 157:105-32 (1982), or in Stryer. The general secondary and tertiary structure of transmembrane domains, in particular the seven transmembrane domains of G protein-coupled receptors such as olfactory receptors, are known in the art. Thus, primary structure sequence can be predicted based on known transmembrane domain sequences, as described in detail below. These transmembrane domains are useful for in vitro ligand-binding assays.

The phrase “functional effects” in the context of assays for testing compounds that modulate OR family member mediated olfactory transduction includes the determination of any parameter that is indirectly or directly under the influence of the receptor, e.g., functional, physical and chemical effects. It includes ligand binding, changes in ion flux, membrane potential, current flow, transcription, G protein binding, GPCR phosphorylation or dephosphorylation, signal transduction receptor-ligand interactions, second messenger concentrations (e.g., cAMP, cGMP IP3, or intracellular Ca.²⁺), in vitro, in vivo, and ex vivo and also includes other physiologic effects such as increases or decreases of neurotransmitter or hormone release.

By “determining the functional effect” or “confirming the activity” in the context of assays is meant assays for a compound that increases or decreases a parameter that is indirectly or directly under the influence of an OR family member, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, oocyte OR gene expression; tissue culture cell OR expression; transcriptional activation of OR genes or activity induced genes such as egr-1 or c-fos; ligand-binding assays; voltage, membrane potential and conductance changes; ion flux assays; changes in intracellular second messengers such as cAMP, cGMP, and inositol triphosphate (IP3); changes in intracellular calcium levels; neurotransmitter release, and the like.

“Binder,” “suppressors,” “blockers,” “inhibitors,” “activators,” and/or “modulators” of OR genes or proteins are used interchangeably to refer to binding, suppressing, blocking, suppressing, inhibitory, activating, or modulating molecules identified using in vivo, in vitro and ex vivo assays for olfactory transduction, e.g., ligands, agonists, antagonists, enhancers, and their homologs and mimetics. Inhibitors are compounds that, e.g., bind to, partially or totally block stimulation, decrease, suppress, prevent, delay activation, inactivate, desensitize, or down regulate olfactory transduction, e.g., antagonists. Activators are compounds that, e.g., bind to, stimulate, increase, open activate, facilitate, enhance activation, sensitize, or up regulate olfactory transduction, e.g., agonists. Modulators include compounds that, e.g., alter the interaction of a receptor with: extracellular proteins that bind activators or inhibitor (e.g., odorant-binding proteins, ebnerin and other members of the hydrophobic carrier family, or a member of the lipocalin family); G proteins; kinases (e.g., homologs of rhodopsin kinase and beta adrenergic receptor kinases that are involved in deactivation and desensitization of a receptor); and arrestins, which also deactivate and desensitize receptors. Modulators also include compounds that alter the affinity or the transduction efficacy of an OR altering the effect of an activator on the OR. Modulators can include genetically modified versions of OR family members, e.g., with altered activity, as well as naturally occurring and synthetic ligands, antagonists, agonists, small chemical molecules and the like. Such assays for inhibitors and activators include, e.g., expressing OR family members in cells or cell membranes, applying putative modulator compounds, in the presence or absence of flavor, fragrance or malodour molecules, e.g. a malodour-causing substance such as DMTS, and then determining the functional effects on olfactory transduction, as described above. Samples or assays comprising OR family members that are treated with a potential activator, inhibitor, or modulator are compared to control samples without the inhibitor, activator, or modulator to examine the extent of modulation. Control samples (untreated with modulators) are assigned a relative OR activity value of 100%. Inhibition of an OR is achieved when the OR activity value relative to the control is about 80%, optionally 50% or 25-0%.

The terms “purified,” “substantially purified,” and “isolated” as used herein refer to the state of being free of other, dissimilar compounds with which the compound of the invention is normally associated in its natural state, so that the “purified,” “substantially purified,” and “isolated” subject comprises at least 0.5%, 1%, 5%, 10%, or 20%, and most preferably at least 50% or 75% of the mass, by weight, of a given sample. In one preferred embodiment, these terms refer to the compound of the invention comprising at least 95% of the mass, by weight, of a given sample. As used herein, the terms “purified,” “substantially purified,” and “isolated” “isolated,” when referring to a nucleic acid or protein, of nucleic acids or proteins, also refers to a state of purification or concentration different than that which occurs naturally in the mammalian, especially human body. Any degree of purification or concentration greater than that which occurs naturally in the mammalian, especially human body, including (1) the purification from other associated structures or compounds or (2) the association with structures or compounds to which it is not normally associated in the mammalian, especially human, body, are within the meaning of “isolated.” The nucleic acid or protein or classes of nucleic acids or proteins, described herein, may be isolated, or otherwise associated with structures or compounds to which they are not normally associated in nature, according to a variety of methods and processes known to those of skill in the art.

As used herein, the terms “amplifying” and “amplification” refer to the use of any suitable amplification methodology for generating or detecting recombinant of naturally expressed nucleic acid, as described in detail, below. For example, the invention provides methods and reagents (e.g., specific degenerate oligonucleotide primer pairs, oligo dT primer) for amplifying (e.g., by polymerase chain reaction, PCR) naturally expressed (e.g., genomic DNA or mRNA) or recombinant (e.g., cDNA) nucleic acids of the invention in vivo, ex vivo or in vitro.

The term “7-transmembrane receptor” means a polypeptide belonging to a superfamily of transmembrane proteins that have seven domains that span the plasma membrane seven times (thus, the seven domains are called “transmembrane” or “TM” domains TM I to TM VII). The families of olfactory and certain taste receptors each belong to this super-family. 7-transmembrane receptor polypeptides have similar and characteristic primary, secondary and tertiary structures, as discussed in further detail below.

The term “nucleic acid” or “nucleic acid sequence” refers to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating, e.g., sequences in which the third position of one or more selected codons is substituted with mixed-base and/or deoxyinosine residues.

In addition to the gene sequences shown in the sequences disclosed herein, it will be apparent for the person skilled in the art that variants also include DNA sequence polymorphisms that may exist within a given population, which may lead to changes in the amino acid sequence of the polypeptides disclosed herein. Such genetic polymorphisms may exist in cells from different populations or within a population due to natural allelic variation. Allelic variants may also include functional equivalents.

Further embodiments also relate to the molecules derived by such sequence polymorphisms from the concretely disclosed nucleic acids. These natural variations usually bring about a variance of about 1 to 5% in the nucleotide sequence of a gene or in the amino acid sequence of the polypeptides disclosed herein. As mentioned above, the nucleic acid encoding the polypeptide of an embodiment herein is a useful tool to modify non-human host organisms or host cells intended to be used in the methods described herein.

The terms “polypeptide,” “peptide” and “protein” are used interchangeably herein to refer to a polymer of amino acid residues, whether comprising naturally occurring amino acids or polymers and non-naturally occurring amino acids or polymers. The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant” means also the ligation of nucleic acids having various coding regions or domains or promoter sequences from different sources into an expression cassette or vector for expression of, e.g., inducible or constitutive expression of a fusion protein comprising a translocation domain of the invention and a nucleic acid sequence potentially amplified using a primer. “Recombinant” means also modifications obtained by genome editing techniques, such as CRISPR/Cas9, of a cell that leads to stable or transient expression of endogenous genes such as the receptor gene referred to herein.

The term “expression vector” refers to any recombinant expression system for the purpose of expressing a nucleic acid sequence of the invention in vitro, ex vivo, or in vivo, constitutively or inducibly, in any cell, including prokaryotic, yeast, fungal, plant, insect or mammalian cell. The term includes any linear or circular expression systems including but not limited to viral vectors, bacteriophages and plasmids. The skilled person is capable of selecting a suitable vector according to the expression system. The term includes expression systems that remain episomal or integrate into the host cell genome. The expression systems can have the ability to self-replicate or not, i.e., drive transient expression in a cell. The term includes recombinant “expression cassettes” which can contain the minimum elements needed for transcription of the recombinant nucleic acid. The term also covers cassettes or vectors for expression of endogenous genes through, for example, genome editing methods such as CRISPR/Cas9.

By “a non-human organism or a host cell” is meant a non-human organism or a cell that contains a nucleic acid as described herein or an expression vector and supports the replication or expression of the expression vector. Host cells may be prokaryotic cells such as E. coli, or eukaryotic cells such as yeast, insect, amphibian, or mammalian cells such as CHO, HeLa, HEK-293, and the like, e.g., cultured cells, explants, and cells in vivo.

By “tag” or “tag combination” is meant a short polypeptide sequence that can be added to the odorant receptor protein. Typically, the DNA encoding a “tag” or a “tag combination” is added to the DNA encoding the receptor, eventually resulting in a fusion protein where the “tag” or a “tag combination” is fused to the N-terminus or C-terminus of the receptor. Lucy, FLAG® and/or Rho tags can enhance the receptor trafficking to the cell membrane, hence the can assist in expression of a functional odorant receptor for in vitro cell based assay [Shepard, B. et al. PLoS One 8, e68758-e68758 (2013), and Zhuang, H. & Matsunami, H. J. Biol. Chem. 282, 15284-15293 (2007)].

In one embodiment provided herein is an isolated nucleic acid molecule comprising a nucleic acid sequence having at least 70%, 75%, 80%, 85%, 90%, 95%, 96%, 97%, 98%, 99%, or 100% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 39, SEQ ID NO: 31, SEQ ID NO: 33, SEQ ID NO: 35, SEQ ID NO: 37, SEQ ID NO: 39 or the reverse complement thereof.

In one embodiment provided herein is an isolated nucleic acid sequence as described above which encodes a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40.

In a further embodiment provided herein is an isolated polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40.

In one embodiment, a non-human organism or a host cell is transformed to express a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40.

In one embodiment, a non-human organism or a host cell is transformed to express a polypeptide comprising an amino acid sequence that is identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40.

Further provided herein is an expression vector comprising a nucleic acid that encodes a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40.

Also provided herein is an expression vector comprising a nucleic acid that comprises a nucleic acid sequence having at least 70%, 75%, 80%, 85%, 90%, 95%, 96%, 97%, 98%, 99%, or 100% sequence identity to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 39, SEQ ID NO: 31, SEQ ID NO: 33, SEQ ID NO: 35, SEQ ID NO: 37, SEQ ID NO: 39 or the reverse complement thereof.

Also provided herein is an expression vector having a nucleic acid wherein the nucleic acid comprises a nucleotide sequence that is identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 39, SEQ ID NO: 31, SEQ ID NO: 33, SEQ ID NO: 35, SEQ ID NO: 37, SEQ ID NO: 39 or the reverse complement thereof.

In one embodiment herein is a method for identifying a compound that binds, suppresses, blocks, inhibits, and/or modulates the activity of an olfactory receptor that is activated by a malodor-causing substance such as dimethyl trisulfide (DMTS) or dimethyl disulfide (DMDS), wherein the receptor is a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40, wherein the method comprises:
a) contacting the receptor, or a chimera or fragment thereof with a compound;
b) determining whether the compound has an effect on the activity of the receptor.

In one embodiment is a method for identifying a compound that binds, suppresses, blocks, inhibits, and/or modulates the activity of an olfactory receptor that is activated by a malodor-causing substance comprising:
a. contacting a test substance and a malodor-causing substance to at least one olfactory receptor selected from the group consisting of Olfr1193, Olfr1093, Olfr1097, Olfr166, Olfr169, Olfr738, Olfr742, Olfr207, Olfr665, Olfr669, Olfr1211, OR52N5, OR2L13, OR2AJ1, OR4C15, OR5AC2, OR8H3, OR11G2, OR52N2, and OR5T1;
b. measuring the response of the olfactory receptor to the malodor-causing substance by measuring the response of the olfactory receptor in the presence and absence of the test substance;
c. identifying a test substance that modulates the response of the olfactory receptor on the basis of the response that was measured in the presence and absence of the test substance; and
d. selecting the identified test substance as a compound that modulates the response of the olfactory receptor to the malodor-causing substance, wherein the malodor-causing substance is dimethyl trisulfide (DMTS) or dimethyl disulfide (DMDS).

In a further embodiment herein is a method for identifying a malodor modulator comprising:
a. contacting a test substance and a malodor-causing substance to at least one olfactory receptor, wherein the receptor comprises a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40;
b. measuring the response of the olfactory receptor polypeptide to the malodor-causing substance;
c. identifying, based on the measured response, a test substance that can modulate or suppress the response of the olfactory receptor; and
d. selecting, as a malodor modulator, the test substance that that binds, suppresses, blocks, inhibits, and/or modulates the response of the olfactory receptor;
wherein the malodor-causing substance is dimethyl trisulfide (DMTS) or dimethyl disulfide (DMDS).

One embodiment is a method for identifying a malodor inhibitor comprising:
a. contacting a test substance and a malodor-causing substance to at least one olfactory receptor, wherein the receptor comprises a polypeptide comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40;
b. measuring the response of the olfactory receptor polypeptide to the malodor-causing substance;
c. identifying, based on the measured response, a test substance that can suppress the response of the olfactory receptor; and
d. selecting, as a malodor inhibitor, the test substance that suppresses the response of the olfactory receptor,
   wherein the malodor-causing substance is DMTS and the malodor is a fecal malodor or oral malodor.

One embodiment is a method of identifying a compound that putatively modulates DMTS associated malodor comprising: (i) contacting a cell line that expresses a DMTS receptor polypeptide comprising an amino acid sequence having at least 90% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40 with at least one compound; (ii) screening for compounds that bind, suppress, block, inhibit, and/or modulate the activity of said olfactory receptor polypeptide; and (iii) identifying a compound that putatively modulates DMTS associated malodor if it binds, suppresses, blocks, inhibits, and/or modulates the activity of said DMTS receptor polypeptide.

In a further embodiment, the malodor-causing substance in the methods described herein is dimethyl trisulfide (DMTS).

Additionally provided is a a recombinant nucleic acid molecule comprising
a. a nucleic acid comprising a tag combination that comprises one or more of a Lucy, FLAG®, and/or Rho tag; and
b. a nucleic acid encoding a receptor selected from the group consisting of Olfr1193, Olfr1093, Olfr1097, Olfr166, Olfr169, Olfr738, Olfr742, Olfr207, Olfr665, Olfr669, Olfr1211, OR4S2, OR52N5, OR2L13, OR2AJ1, OR4C15, OR5AC2, OR8H3, OR11G2, OR52N2, and OR5T1 or the complement thereof.

In a further embodiment the Lucy tag comprises SEQ ID NO: 47, the FLAG® tag comprises SEQ ID NO: 43, and the Rho tag comprises SEQ ID NO: 45.

Further provided is any one of a number of malodor modulating compounds that binds, suppresses, blocks, inhibits, and/or modulates the activity of an olfactory receptor that is activated by a malodor-causing substance such as dimethyl trisulfide (DMTS) and that is identified by the methods disclosed herein.

In one embodiment herein is a malodor modulating compound that binds, suppresses, blocks, inhibits, and/or modulates the activity of at least one olfactory receptor selected from the group consisting of Olfr1193, Olfr1093, Olfr1097, Olfr166, Olfr169, Olfr738, Olfr742, Olfr207, Olfr665, Olfr669, Olfr1211, OR52N5, OR2L13, OR2AJ1, OR4C15, OR5AC2, OR8H3, OR11G2, OR52N2, and OR5T1, and that is identified by the methods disclosed herein.

Another embodiment relates to the use of a polypeptide that is or can be activated by DMTS or DMDS comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, or SEQ ID NO: 40 for identifying a malodor modulating compound that binds, suppresses, blocks, inhibits, and/or modulates the activity of an olfactory receptor.

Still yet further provided is a cell that is recombinantly modified to express a polypeptide described above.

In one embodiment herein is a non-human host organism or a host cell that has been transformed or modified to express a receptor that is activated by DMTS selected from the group consisting of Olfr1193, Olfr1093, Olfr1097, Olfr166, Olfr169 Olfr738, Olfr742, Olfr207, Olfr665, Olfr669, Olfr1211, OR4S2, OR52N5, OR2L13, OR2AJ1 OR4C15, OR5AC2, OR8H3, OR11G2, OR52N2, and OR5T1.

In one embodiment herein is is a non-human host organism or a host cell that has been transformed or modified to express a receptor that is activated by DMTS, wherein the receptor comprises a polypeptide as described herein or a polypeptide encoded by a nucleic acid described herein.

In a further embodiment herein is a non-human host organism or a host cell that comprises a nucleic acid or expression vector as described herein.

In one embodiment provided herein is a cell wherein the cell is a prokaryotic cell. In another embodiment the cell provided herein is a eukaryotic cell. In a particular embodiment, the cell provided herein is selected from a group consisting of a yeast cell and a plant cell. In a more particular embodiment provided herein the cell is selected from the group consisting of HEK293, CHO, Xenopus oocytes, COS, yeast, bacteria and cells derived from the olfactory placode.

In order to identify unknown DMTS or DMDS specific receptors, DMTS and DMDS can be used to screen dissociated olfactory sensory neurons (OSNs). DMTS and DMDS can be further used for cell-based dose-response experiments performed on specific DMTS or DMDS receptors to assess both specificity and sensitivity of the receptors.

In one aspect, provided herein are methods to identify mammalian odorant receptors for malodour modulating compounds and the use of the receptors for screening, particularly for high throughput screening (HTS) of malodor modulators (e.g. that bind, suppress, block, inhibit and/or modulate the activity of an OR).

In particular provided herein are mouse receptors, for example, Olfr1193, Olfr1093 Olfr1097 Olfr166 Olfr169, Olfr738 Olfr742 Olfr669, Olfr665 Olfr207, Olfr1211, and their human counterparts OR4S2, OR5T1, OR8H3, OR2L13, OR2AJ1, OR11G2, OR52N5, OR52N2, OR5AC2, and OR4C15, as receptors for the malodour-causing substances DMTS, DMDS or other polysulfide compounds, as shown in Table 1.

TABLE 1
Orthologous mouse and human odorant receptor pairs and
their corresponding identity at the amino acid level.

| Mouse ortholog | Human ortholog | Amino acid identity |
|---|---|---|
| Olfr1193 | OR4S2 | 90% |
| Olfr1093 | OR5T1 | 77% |
| Olfr1097 | OR8H3 | 70% |
| Olfr166 | OR2L13 | 85% |
| Olfr169 | OR2AJ1 | 72% |
| Olfr738 | OR11G2 | 79% |
| Olfr742 | OR11G2 | 74% |
| Olfr669 | OR52N5 | 87% |
| Olfr665 | OR52N2 | 77% |
| Olfr207 | OR5AC2 | 64% |
| Olfr1211 | OR4C15 | 83% |

While not wishing to be bound to any theory, mouse receptor Olfr738 is a paralog of receptor Olfr742 and human OR11G2 is the ortholog of these mouse receptors; and mouse receptor Olfr669 is a paralog of receptor Olfr665 and an ortholog of human receptor OR52N5; and mouse receptor Olfr665 is an ortholog of human receptor OR52N2. Several receptors were previously identified as indole and/or skatole responsive odorant receptors in WO2014/210585. However, these receptors had not been previously associated with the malodour-causing substances DMTS or DMDS or other related polysulfide compounds. Indole and skatole are not chemically related to polysufide compounds and therefore approaches based on chemical structure such as structure-activity-relationship (SAR) to identify additional ligands would not define DMTS as a potential agonist of these receptors.

In a further embodiment, indicators for monitoring the activity of olfactory receptors are selected from a fluorescent calcium indicator dye, a calcium indicator protein, a fluorescent cAMP indicator, a cell mobilization assay, a cellular dynamic mass redistribution assay, a label-free cell based assay, a cAMP response element (CRE) mediated reporter protein, a biochemical cAMP HTRF assay, a beta-arrestin assay, or an electrophysiological recording. Particularly, a calcium indicator dye is selected that can be used to monitor the activity of olfactory receptors expressed on the membrane of the olfactory neurons (e.g., Fura-2 AM).

In a particular embodiment, compounds are screened sequentially and the odorant-dependent changes in calcium dye fluorescence are measured using a fluorescent microscope or fluorescent-activated cell sorter (FACS).

In a further embodiment, molecular 3D receptor modeling of olfactory receptors is used to assess the binding potential in silico and to identify compounds that may activate, mimic, block, inhibit, modulate, and/or enhance the activity of an olfactory receptor.

As an example, olfactory neurons activated by DMTS or DMDS are isolated using either a glass microelectrode attached to a micromanipulator or a FACS machine. Mouse olfactory sensory neurons are screened by Ca²⁺ imaging similar to procedures previously described [Malnic, B., et al. Cell 96, 713-723 (1999); Araneda, R. C. et al. J. Physiol. 555, 743-756 (2004); and WO2014/210585 hereby incorporated by reference in its entirety]. Particularly, a motorized movable microscope stage is used to increase the number of cells that can be screened to at least 1,500 per experiment. Since there are approximately 1,200 different olfactory receptors in the mouse and each olfactory sensory neurons expresses only 1 of 1,200 olfactory receptor genes, this screening capacity will cover virtually the entire mouse odorant receptor repertoire. In other words, the combination of calcium imaging for high-throughput olfactory sensory neuron screening leads to the identification of nearly all of the odorant receptors that respond to a particular profile of odorants. In a particular aspect, odorant receptors that respond to DMTS or DMDS can be isolated. For example, at least one neuron is isolated.

For calcium imaging of olfactory neurons, the main olfactory epithelium may be dissected from a mouse before neuronal dissociation. Dissected olfactory epithelium may then be transferred to a dissociation buffer for mechanical and enzymatic dissociation. Dissociated neurons may then be seeded onto a coverslip allowing the screening of thousands of cells by fluorescence microscopy and the cells may be loaded with a calcium sensitive dye (Fura-2 AM) for example for about 30 minutes at 31° C. and transferred onto the microscope ready for screening. Cells are stimulated by perfusing diluted solutions of odorants (in physiological saline) over the dissociated olfactory neurons. The rare cells that respond to the malodor compound are identified by for example stimulating the receptors with 50 μm of the malodor compounds and then by monitoring the intracellular Ca²⁺ flux indicated by changes in Fura-2 fluorescence. After analysis, responding cells may be retrieved from a glass coverslip with a suction micropipette. Isolated cells are then pooled into one sample or treated individually for subsequent identification of the odorant receptor genes expressed as mRNA in the responding cells.

In a particular embodiment, the mRNAs of olfactory neurons are purified and amplified according to the method generally described in Marko, N. F., et al., (2005) A robust method for the amplification of RNA in the sense orientation. BMC genomics, 6, 27; doi:10.1186/1471-2164-6-27 (Eberwine method). At least a portion of the transcriptome (up to including the entire transcriptome) is sequenced using Next-Generation Sequencing (NGS) technologies or hybridized to known genes using Microarray technologies. NGS is generally discussed and described in Metzker, M. L. Nat. Rev. Genet. 11, 31-46 (2010). In a particular embodiment, a minimum of 5 neurons presenting the same response profile are pooled. The mRNAs are released by cell lysis immediately after picking; no DNAse and no purification steps are carried out. The mRNA are amplified by two consecutive rounds of in vitro transcription (IVT). The amplification may be done according to MesageAmpII aRNA kit (Ambion, AMA1751) with the following parameters: two rounds of consecutive 14 hour long IVT.

In a further embodiment, the mRNA of a single olfactory neuron is purified and amplified with LD-PCR (Long Distance Polymerase Chain Reaction) based methods such as the one described in NGS-ready kits (e.g., Clontech/Takara, SMARTer® Ultra® Low Input RNA Kit for Sequencing—v3, cat. 634848). Single cell mRNA is first reverse transcribed into the corresponding cDNA, which subsequently is amplified with 18 PCR cycles and serves as NGS sample for transcriptome sequencing.

In yet another embodiment, the identity of a group or gene family of DMTS olfactory receptors is determined (e.g., up to as many as the number of neurons picked) by comparing the results of the NGS reads obtained from the isolated activated olfactory sensory neurons to a reference genome sequence of the same species. Particularly, the putative DMTS receptors will be the most highly abundant olfactory receptor mRNA in the olfactory neuron-derived NGS sample or present in more than one independent biological replicate. Because of the combinatorial nature of the olfactory code (one compound activates many ORs and one OR can be activated by many compounds), pooling several neurons activated by given compounds allows the retrieval of virtually all of the receptors responsible for the perception of these molecules in a single NGS experiment. Pooling functionally similar neurons thus greatly improves the deorphanization throughput and speed.

Standard bioinformatics tools are then used to identify the most closely related human odorant receptor(s) to other putative mammalian (non-human) DMTS receptor(s) under the assumption that homologous sequence receptors retain similar function. Several methods successfully identify human OR-ligand pairs based on this assumption [Armelin-Correa and Malnic (2017)] and up to 80% of mouse-human orthologs appear to maintain similar functional response profiles [Adipietro, K. A, et al. PLoS Genet. 8, e1002821-e1002821 (2012)]. Default parameters of BLASTP and/or BLASTN algorithm, or other ortholog pair identification algorithms such as InParanoid may be used.

The human or non-human mammalian DMTS receptors may be adapted to a functional assay that can be used to identify compounds that bind, suppress, block, inhibit, and/or modulate the activity of the olfactory receptors. In particular, the assay may be a cell-based assay or a binding assay and the method for identifying compounds may be a high-throughput screening assay. More particularly, provided herein are receptor-based assays adaptable for high-throughput screening of receptors with compound libraries for the discovery of modulating compounds (e.g., binding, blocking, inhibiting, suppressing and masking).

In a particular embodiment, DMTS receptor gene sequences are identified from DMTS-sensitive cells as follows: Pooled neurons are heated to 75° C. for 10 minutes to break the cell membrane and render their mRNA available for amplification. This amplification step is important when applying NGS technologies with limited amount of starting material, typically between 1 to 15 cells. A linear amplification according to the Eberwine method (IVT) ensures the maintenance of the relative transcription levels of expressed genes. Two consecutive overnight (14 h) rounds of in vitro transcription are used to yield sufficient amounts of cRNA; Amplified cRNA is then used to generate an Illumina HiSeq cDNA library. The resulting short sequences of typically 75 to 150 base pairs (commonly referred to as “reads”) are aligned against the reference genome of the mouse (such as UCSC version mm9 or mm10) in order to build the full transcriptome of these cells. Quantitative analysis of the transcriptome data yields a list of transcribed odorant receptor genes and their respective expression levels. Odorant receptor genes that show the most abundant levels of mRNA (most abundant “reads”) or are present in more than one replicate experiment are considered putative DMTS receptors.

The predicted mouse OR genes are then used to mine the latest versions of both the mouse and human genome databases in order to identify the most closely related receptors (i.e. highest sequence similarity) in mouse (paralogous genes) and in human (orthologous genes). This process may be performed using the BLAST search algorithm (publically available at the NCBI website), a sequence similarity search tool, where every putative gene sequence previously obtained from the initial transcriptome analysis is used as a query sequence. The newly identified genes identified from this data mining process are considered to be potential DMTS receptors under the assumption that paralogous and orthologous genes are highly likely to possess similar activities. In a particular embodiment, pairwise comparison of sequence homology is carried out to identify closely related receptors in mouse and humans and the receptors are identified as described in WO2014/210585. Other approaches may also be used such as RT-PCR and microarray approaches.

In a further embodiment, to complete the deorphanization process, the candidate OR genes are further expressed in vitro for confirmation of activity against the compounds used to isolate the olfactory sensory neurons and other structurally-related compounds of interest. The mouse receptors identified from isolated olfactory neurons that respond to DMTS are modified at their N-terminus with short polypeptide sequences (e.g., FLAG® (SEQ ID NO: 44), Rho (SEQ ID NO: 46; 20 first amino acids of the bovine rhodopsin receptor), and/or Lucy (SEQ ID NO: 48; cleavable leucine-rich signal peptide sequence) tags), transiently expressed in HEK 293T cells, and stimulated separately with DMTS to confirm their identity as bona fide DMTS receptors. In a further embodiment, an RTP1 gene can also be expressed in the cell lines whether through activation of the endogenous RTP1 gene or through transformation. Co-expression of the human G alpha subunit Gaoif in this cell-based assay activates the Gs transduction pathway that leads to an internal cAMP increase upon binding to the appropriate ligand. Alternatively, co-expression of the human G alpha subunit Gals in the cell based assay activates the Gq transduction pathway that leads to an internal Ca′ increase upon binding to the appropriate ligand. The above process and the results obtained so far serve to validate the process for rapid and reliable identification of mammalian odorant receptors for DMTS or DMDS.

Further provided are assays for identifying compounds that bind to DMTS or DMDS odorant receptors. In a further embodiment provided herein is a malodor modulating compound that binds, suppresses, blocks, inhibits, and/or modulates the activity of at least one olfactory receptor selected from the group consisting of Olfr1193, Olfr1093, Olfr1097, Olfr166, OR52N5, OR2L13, OR4C15, OR5AC2, OR8H3, OR11G2, and OR52N2, and that is identified by the methods described herein, for example, the compounds described in FIG. 4.

In one embodiment the activity of the compound is determined by comparing its binding to that of DMTS or DMDS. In another embodiment, the receptor or a chimera or fragment thereof is contacted with a compound in the presence of DMTS or DMDS under conditions that allow for the binding of the compound along with DMTS or DMDS to the receptor.

In a further embodiment, a compound is contacted to a receptor, or a chimera or fragment thereof that is activated by DMTS or DMDS, wherein the receptor, or a chimera or fragment thereof is expressed in a cell that is recombinantly modified to express the receptor polypeptide.

The activity of the compound can be determined using in vivo, ex vivo, in vitro and synthetic screening systems.

In another embodiment, the contacting is performed with liposomes or virus-induced budding membranes containing the polypeptides described herein.

In another embodiment, the methods for identifying compounds that bind, suppress, block, inhibit, and/or modulate the activity of an olfactory receptor that can be activated by DMTS or DMDS, may be performed on intact cells or a membrane fraction from cells expressing the polypeptides described here or on olfactory sensory neurons in culture modified to express endogenous or exogenous odorant receptors.

The 21 ORs described herein are therefore involved in the perception of malodor elicited by DMTS or DMDS and constitute valuable candidate receptors for the identification of modulators, antagonists and/or blockers that would modulate, reduce, suppress, inhibit and/or block the perception of malodor.

Nucleic acid and amino acid sequences identified and/or used herein are listed below:

Olfr1193
DNA
SEQ ID NO: 1
atggcctcaaggacttattccatggaagaagtaaataatgtcactgaattcattttcttgggtct
ttctcagaaccctgaggttgaaaaagtgtgctttgtggtgttctccttcttttacatggtcattc
tgctaggaaacctcctcatcatgttgacagtttgcagtggcaatcttttcaagtttcccatgtat
tttttcctcaactttctgtcttttgtggacatttgctactcctcagtcacagcacccaagatgat
tattgacctgttagtgaagaaaaagactatatcctatgtggggtgcatgttacaactctttgtgg
ttcatttctttggttgcactgagatcttcattcttactgtcatggcctatgatagatatgtggcc
atttgtaaacctctccactatatgactatgatggaccgggaaagatgcaataagatgttgctcgg
aacatggatcggtggcttcttacattctattatccaagtggctcttgtggtccagctcccctttt
gtggaccgaatgagattgatcactatttctgtgatgtacatcctgtactgaaacttgcctgcact
gacacttacattgttggtatttttgtgacagcaaacagtggcaccattgcattgggaagttttgt
catcttgctgatctcatacacagtcattctcatgtctctgagaaagcagtcatctgaaggcagac
gcaaagctctctccacttgtggatcccacattgctgttgtcatcattttttttggcccctgtact
tttatgtatatgcggcctgacactaccttctctgaggacaagatggtagctatattttacaccat
tatcactcccatgctgaatcctctaatttacactctaagaaatgcagaagtaaagaatgcaatga
gaaaactgtgggctagaaagttttcctgggaaactactgggaaatag
Protein
SEQ ID NO: 2
MASRTYSMEEVNNVTEFIFLGLSQNPEVEKVCFVVFSFFYMVILLGNLLIMLTVCSGNLFKFPMY
FFLNFLSFVDICYSSVTAPKMIDLLVKKKTISYVGCIVLQLFVVHFFGCTEIFILTVMAYDRYVA
ICKPLHYMTMVDRERCNKIVLLGTWGGFLHSIIQVALVVQLPFCGPNEIDHYFCDVHPVLKLACT
DTYIVGIFVTANSGTIALGSFVILLISYTVILMSLRKQSSEGRRKALSTCGSHIAVVIIFFGPCT
FMYMRPDTTFSEDKMVAIFYTIITPMLNPLIYTLRNAEVKNAMRKLWARKFSWETTGK
Olfr1093
DNA
SEQ ID NO: 3
atggaaaagatcacatcagctgtggatgtccacaatattccattaaagaacatgactgaagccac
catgtttattctcttaggattcacagatgactttgaactccaagtcttcctgtttttactgtttc
ttgctatttatctcttcactctggtaggaaactttggactggttgttttggtcattggggattgt
cggctacacaaccccatgtactatttcctaagtgttttgtctttcctggatgcttgctattctac
agttgttacacccaaaatgttggtcaactttctaagtgaaaataagtccatttcattccttgcat
gtgcaacccaaatgcttctctttgtttcgttgggaaccacagaatgctttctcctggcagcaatg
gcttatgaccgatatgtagccatctacaacccacttctgtatacagtggccatgtcacccagagt
atacctgccactcatcattgcttcctatgctggtggagttgtgcatggtgctatccacacagtgg
ccactttcagtctgtccttctgtggatccaatgaaattaagcatgtcttctgtgacatccctgca
ttgcttgctctttcttgttctgatacccacacaaatgagcttctagtcttgtacttggtgggctt
gattgagattgttaccatcctgattgttctggtctcctatggattcatcctctttgccattctga
acatgcattctgctgagggtaggaggaaagtgttctctacatgtggctctcacctcactggagtc
tctatttaccatggtacaatccttttcacttatatgaggcctagttccagttatgcttcaaatca
tgacatggtagtgtcaatattttacaccattgtgatacccatgttgaatcctatcatctatagtt
tgaggaacaaagatgtaaaagtagcatttaataaattgtggagaaaatgtgattcataa
Protein
SEQ ID NO: 4
MEKITSAVDVHNIPLKNMTEATMFILLGFTDDFELQVFLFLLFLAIYLFTLVGNFGLVVLVIGDC
RLHNPMYYFLSVLSFLDACYSTVVTPKMLVNFLSENKSISFLACATQMLLFVSLGTTECFLLAAM
AYDRYVAIYNPLLYTVAMSPRVYLPLIIASYAGGVVHGAIHTVATFSLSFCGSNEIKHVFCDIPA
LLALSCSDTHTNELLVLYLVGLIEIVTILIVLVSYGFILFAILNMHSAEGRRKVFSTCGSHLTGV
SIYHGTILFTYMRPSSSYASNHDMVSIFYTIVIPMLNPIIYSLRNKDVKVAFNKLWRKCDS
Olfr1097
DNA
SEQ ID NO: 5
atgagtgcctgtaatcatacaaatgaacctgagttcacgcttgtgggactgacagactccaagga
gattcagctggtcctctctgttttgtttctcctgatatacatgctcactgtcttgggaaacatag
gtatgatactgatcattcatctagatgtccagctccacactccaatgtattttttcctcacccac
ttgtcattccttgacctcagttactcaactgtaatcacacctaaaaccttacagaatacgctgac
ctccataaaaaatatttccttcatgggatgcttcacccagttgtatttctttgtcctcttggcag
cttctgaatgttttatactttcgtcaatggcctatgaccgctatgtagctatctgcaaccctcta
cactatccagttattatgtcccctaggcgctcatatactctcatcactgtgtcctacatgattgg
agttttggattcttctgtcactgtcttttgcttaagcacactggatttctgcaactccaaagtaa
ttcatcacttcttttgtgacacattcccaattttagctctgtcctgcagtgatacctataatgca
gaagccactatattcgttttagctggttccactctattgctgtcgctcatcacgatatcctcatc
ctatgtatctattctctctacaattttgaagataaattcttcttcaggaaagcacaaagccttct
ctacatgtgcctcacatcttataggagtcactgttttttatggtacaatgatctttacttattta
aaaccaagtacgtcctactccctgggaaaggatcaagtagcctctgttttttatactatagtgat
tcccatgctgaacccacttatctatagtctcaggaacaaagaagtgaaaagtgctgttgttagag
ttatgaagaagagagagtgcatccagaaactagaataa
Protein
SEQ ID NO: 6
MSACNHTNEPEFTLVGLTDSKEIQLVLSVLFLLIYIVLTVLGNIGMLIIHLDVQLHTPMYFFLTH
LSFLDLSYSTVITPKTLQNTLTSIKNISFMGCFTQLYFFVLLAASECFILSSMAYDRYVAICNPL
HYPVIMSPRRSYTLITVSYMIGVLDSSVTVFCLSTLDFCNSKVIHHFFCDTFPILALSCSDTYNA
EATIFVLAGSTLLLSLITISSSYVSILSTILKINSSSGKHKAFSTCASHLIGVTVFYGTMIFTYL
KPSTSYSLGKDQVASVFYTIVIPMLNPLIYSLRNKEVKSAVVRVMKKRECIQKLE
Olfr166
DNA
SEQ ID NO: 7
atggagaaatggaatcagagctcaagtgattttactctgttaggactgcttccacaaaaccaaac
aggcctgctacttttgatgctcatcatctttgtcttctctctggctttgtgtggcaactcaggaa
tgatccacctcattcgtgtggatccaaggctccacacccccatgtactttctcctcagtcagctc
tctctcatggacctgatgtacatttctaccactgttcccaagatggcatttaacttcctttctgg
ccagaaaagcatctcttttctgggctgtggagtgcaatccttcttcttcctgactatggcatgtt
ctgagggcttgctcttggcttccatggcttatgatcgttttgtggctatctgccatccccttcac
tatcccattcgcatgagcaaaataatgtgtctgaagatgatcataggatcctggatattgggctc
aatcaactctttagcacataccgtctatgcccttcatattccttactgccattctaggtccatta
accatttcttctgtgatgttccagccatgttgcccctggcctgtatggacacttgggtttatgag
tacatggtgtttgtgagcacaagcctgtttctcctactgcctttccttggtatcacagcttccta
tggtcgggtcctttttgctgtcttccacatgcgctcaaaagagggaaagaagaaggccttcacca
catgctcaactcacttaactgtggtgacattttactatgcaccttttgtctatacctatcttcga
cctaggagtcttcgctccccaacagaagataagattctggctgttttctacactatccttacccc
catgctcaaccccatcatttatagtctgaggaataaggaggtcctgggggccatgacaagagtcc
ttggtacttttccttcaactaaaccgtaa
Protein
SEQ ID NO: 8
MEKWNQSSSDFTLLGLLPQNQTGLLLLMLIIFVFSLALCCNSGMIHLIRVDPRLHTPMYFLLSQL
SLMDLMYISTTVPKMAFNFLSGQKSISFLGCGVQSFFFLTMACSEGLLLASMAYDRFVAICHPLH
YPIRMSKIMCLKMIGSWILGSINSLAHTVYALHIPYCHSRSINHFFCDVPAIVLPLACMDTWVYE
YMVFSTSLFLLLPFLGITASYGRVLFAVFHIVRSKEGKKKAFTTCSTHLTVVTFYYAPFVYTYLR
PRSLRSPTEDKILAVFYTILTPMLNPIIYSLRNKEVLGAMTRVLGTFPSTKP
Olfr169
DNA
SEQ ID NO: 9
atggaatatgagaactacacttttaacagcgacttcatcctcttgggactgttctcttcttcaaa
gacaagcttaacttttttctcatttatatttttcatttttattatggctataacagaaaatgccc
tcatgatcctcctaatccacagggattctcgactccataccccaatgtatttcctgcttagtcat
ctctccttcatggatatcttgcacatttccaacattgttcctaaaatgattgctgacttcctctc
aggcagcagaactatttcctttgcaggctgtgccttccagatatttctctctcttaccttgctag
gtggtgagtgccttctcctggcagccatgtcctatgatcgatatgtggccatctgccacccactt
cgctaccctgtgctgatgagggataactccagtaggctcctggctgcaggctcctggctggtggg
gatcctcaactccatagtacacacagtttttgcactccactttcccttctgccactcaagagcca
ttgatcactttttctgcgaagtccctgccatgttgaaattgtcatgtatagacacaacacactat
gaacgaggcgtttatgtgagtggcattatttttctgctgatcccattttccatgatctctatatc
ttatgtgcaaattctcctcactgtattccaaatgcagtcatcaggggcccggcaaaagtcctttt
ccacctgttccttccacatggttgttgtcataatgtactatgggccattcatttttacatatatg
agacctcgctcataccacactccagggcaggataaatttttggcaatattctacaccatcctgac
acccacactcaaccccataatctacagctttcgtaataaagatgtccttatggctgtgaaaaaca
tcgtccaaagtaattttttgaataaaaaatga
Protein
SEQ ID NO: 10
MEYENYTFNSDFILLGLFSSSKTSLTFFSFIFFIFIMAITENALMILLIHRDSRLHTPMYFLLSH
LSFMDILHISNIVPKMIADFLSGSRTISFAGCAFQIFLSLTLLGGECLLLAAMSYDRYVAICHPL
RYPVLMRDNSSRLLAAGSWLVGILNSIVHTVFALHFPFCHSRAIDHFFCEVPAMLKLSCIDTTHY
ERGVYVSGIIFLLIPFSMISISYVQILLTVFCMQSSGARQKSFSTCSFHMVVVIMYYGPFIFTYM
RPRSYHTPGQDKFLAIFYTILTPTLNPIIYSFRNKDVLMAVKNIVQSNFLNKK
Olfr738
DNA
SEQ ID NO: 11
atgaaagcctttagcagccccagcaactccagcatcatcactggcttcatcctcctgggcttccc
ctgccccaaggaggggcaaatcctcctctttgtgctcttcttcattatctacatccttaccctca
tgggcaatgcttccatcatatgtgctgtgtgctatgataagaaacttcacagccccatgtacctc
ctgctggccaacttctccttcctagaaatctggtatgtcacctccacagtccccaacatgttggc
caacttcctctctgacacgaaggtcatctctttctctggatgcttcctgcagttctatttcttct
tctccttgggttctacagaatgctttttcctggcagtcatggcatttgatcgataccttgccatc
tgcagacctctacattatccttctctcatgactgggcgcctctgcaacatccttgtgatcagttg
ctgggtgcttggtttcctctggttccctgttcccatcatcatcatctcccaaatgtccttctgtg
gatccagaattatagaccacttcctgtgtgacccaggccctctgttggccctcacctgtgtgaga
aattctttaattgagatgactagctctactttaagttccctgcttttatttgttccatttttttt
tatcatggggtcttatgctctagtaatgagggctgtgctcagggtcccttcagcagctggacgaa
gaaaggccttctccacctgtgggtcacacttgactgtggtttctcttttctatggctcagtgatg
gtcatgtatgtgagcccaacatctgaacatgcagctggagtgcaaaaacttgtgactctgtttta
ttctgtggttactcccctccttaatcctgtgatatacagtctgaggaacagagatatgaaacatg
caatgaaaaagttactgaaaatgtaa
Protein
SEQ ID NO: 12
MKAFSSPSNSSIITGFILLGFPCPKEGQILLFVLFFIIYILTLMGNASIICAVCYDKKLHSPMYL
LLANFSFLEIWYVTSTVPNMLANFLSDTKVISFSGCFLQFYFFFSLGSTECFFLAVMAFDRYLAI
CRPLHYPSLMTGRLCNILVISCWVLGFLWFPVPIIIISQMSFCGSRIIDHFLCDPGPLLALTCVR
NSLIEMTSSTLSSLLLFVPFFFIMGSYALVMRAVLRVPSAAGRRKAFSTCGSHLTVVSLFYGSVM
VMYVSPTSEHAAGVQKLVTLFYSVVTPLLNPVIYSLRNRDMKHAMKKLLKM
Olfr742
DNA
SEQ ID NO: 13
Atgaaaaccctcagcagccccagcaactccagcaccatcactggcttcatcctcttgggcttccc
ctgccccagggaggggcaaatcctcctctttgtgaccttcttcattgtttacatactcattctta
tgggcaatgcttccatcatctgtgctgtgtactgtgatcagagcctccacacccccatgtacttc
ctgctggccaacttctccttcctggagatctggtatgtcacctccacagtccccaacatgttggc
caacttcctttcagacaccaaggtcatctctttctctggatgcttcctgcagttctatttcttct
tctcctttggttctacagaatgctttttcctggcagtcatggcatttgatcgataccttgccatc
tgtaggccactacattatccttctctcatgactgggcacctctgcaacatccttgtgatcagttg
ctgggtgcttggtttcctctggttccctgtacccatcatcatcatctcccagatgtccttctgtg
ggtccagaattatagaccacttcctgtgtgacccaggccctcttttggcccttgcctgttccaga
gccccattgatggaggttttctggacaattataatgtctatgctcctggttattcctttcctctt
catcatgggaacttacatattggtcctaagagctgtgtttagacttccttcaagagatggacaaa
aaaaggccttctccacttgcgggtctcatctcacagtagtttcactcttttattgctcagtgatg
aaaatgtatttgagcccaacatctgagcatgaagctggaatgcagaagcttgtaactctatttta
ttctgtgggtactccactacttaatcctgtgatatacagtctgaggaacaaagatatgaaaaatg
ccctgcagaagattttaagaacataa
Protein
SEQ ID NO: 14
MKTLSSPSNSSTITGFILLGFPCPREGQILLFVTFFIVYILILMGNASIICAVYCDCSLHTPMYF
LLANFSFLEIWYVTSTVPNMLANFLSDTKVISFSGCFLQFYFFFSFGSTECFFLAVMAFDRYLAI
CRPLHYPSLMTGHLCNILVISCWVLGFLWFPVPIIIISQMSFCGSRIIDHFLCDPGPLLALACSR
APLMEVFWTIIMSMLLVIPFLFIMGTYILVLRAVFRLPSRCGQKKAFSTCGSHLTVVSLFYCSVM
KMYLSPTSEHEAGMQKLVTLFYSVGTPLLNPVIYSLRNKDMKNALQKILRT
Olfr207
DNA
SEQ ID NO: 15
atggaactgaacaggacccagctgactgaatttgttctcagaggaataacagatcgttcagagct
gcaagtccccctgttcctggtgttctttctcatctatgttatcaccatggtgggcaaccttggct
taatctttgtcatctggaaggaccctcatcttcacacacccatgtaccttttccttggaaatttg
gcctttgctgatgcctgtaattcatcctctgtgacaccaaagatgcttatgaaatttttaaataa
gaatgacatgatatccatgggtgagtgttttgctcaattttatttcttttgttcaagtgtaactg
cagaagccttcattctggtagctatggcctatgaccgctatgtagccatatgcaaacctctgctc
tatgtagtggtgatgtccaacagactctgtattcagttcataggtgtatcctatctaattggact
tctacatggcttacttcatgtaggattgttatttaggttaacgttttgtagttccaatgtaatag
attatttctactgtgacatcctgccactttataggatttcttgcactgacccatcgatcaatgta
ctggtagctttcattatgggtattttattacaagtgagtacctttatgagtattatagtctccta
tgtccgtgtcctctttgccatcctgagaacaaagtctgagaggggcagaaacaaagccttctcta
cttgcagttcccacctgtcatctgtgtctttgttctatggcactctcttcatcatatatgtcctc
tctggctctgacacagataattatcagggtaaaatgtattcactgttctataccattatcattcc
tctgctaaaccccttcatttacagcctaagaaataaagaagtcatcggtgccttgagaaaagtca
gaaaatga
Protein
SEQ ID NO: 16
MELNRTQLTEFVLRGITDRSELQVPLFLVFFLIYVITMVGNLGLIFVIWKDPHLHTPMYLFLGNL
AFADACNSSSVTPKMLMKFLNKNDMISMGECFAQFYFFCSSVTAEAFILVAMAYDRYVAICKPLL
YVVVMSNRLCIQFIGVSYLIGLLHGLLHVGLLFRLTFCSSNVIDYFYCDILPLYRISCTDPSINV
LVAFIMGILLQVSTFMSIIVSYVRVLFAILRTKSERGRNKAFSTCSSHLSSVSLFYGTLFIIYVL
SGSDTDNYQGKMYSLFYTIIIPLLNPFIYSLRNKEVIGALRKVRK
Olfr665
DNA
SEQ ID NO: 17
atgcctggggtcaatacctccagcctgacaccaagatactttattctcaatgggattcctgggtt
ggaagctgcacacatctggatctctctgccattcttcattatgtacctcattgctgtcacaggta
actgtggacttatctacctcatcagtcatgaggaggctctgcaccggcccatgtactactttcta
gccatgttgtctgctacagatatttctgggtgtaatacaattgtccccagtatgttatgcatctt
ttggttcagtgtcaaggagattgatttcaatgcctgccttgtacagatgtttttcatccacatgt
taacaggcatggagtctggtgtgctcatgcttatggctctcgaccgctatgtggctatatgctat
ccattacgctatactaccatactcaccaacactatgattaccaagattggattggcagcacttgt
tagaagtgtgttactcatggtcccttttgctttcctgatcaagcgtcttccatactgtagaggaa
acctcatccaacatacctattgtgatcacatggctgtggctaaactatcctgtggcaatattaag
attaatgctatctatggtcttataattgctatatttattgggggttttgatatattctgtatctc
catgtcttatgccatgattatccatgctgtggtgaagctatcttcggcagatgctcgccataaag
ccttcagtacctgtacatcacacatatgtgctattgttattacctatgtcccagcattcttcaac
ttctttactcatcgctttgggagaaccactataccccatcatatccacattattatagccaacct
gtatctattgctacctcccaccttgaatccaattgtatatggagtaaagaccaagcagattcgtg
aaggtgtgatcaaactgtttgctagacaaaaagttgtttga
Protein
SEQ ID NO: 18
MPGVNTSSLTPRYFILNGIPGLEAAHIWISLPFFIMYLIAVTGNCGLIYLISHEEALHRPMYYFL
AMLSATDISGCNTIVPSMLCIFWFSVKEIDFNACLVQMFFIHMLTGVESGVLMLMALDRYVAICY
PLRYTTILTNTMITKIGLAALVRSVLLMVPFAFLIKRLPYCRGNLIQHTYCDHMAVAKLSCGNIK
INAIYGLIIAIFIGGFDIFCISMSYAMIIHAVVKLSSADARHKAFSTCTSHICAIVITYVPAFFN
FFTHRFGRTTIPHHIHIIIANLYLLLPPTLNPIVYGVKTKQIREGVIKLFARQKVV
Olfr669
DNA
SEQ ID NO: 19
atgctgatttccaacaactcatatgaagccccgcagtctttcattcttaatggaattcctggtct
cgaagcagtgcatatatggatctctcttccactctgtacaatgtacatcatctccctagtaggca
accttggccttgtatatctcatttactatgaggaatccttacatcgcccaatgtatttctttctg
gccatgctttctctcatagacctgtttacttgcacaaccactgtccccaatgccctcttcatttt
ctggttcaaactcaaggaaattaacttcactgcttgcctagttcagatgttctttgtgcacggat
tcacaggtgtggagtctggggtactcatgctcatggccttggaccgctatgtggccatttgctac
ccactacgctatgcaaccatacttaccaaccctgtcattgccaaagctgggcttgccaccttctt
gagaggtgtgttactgatgattccttttccattcttggttaaacgtttgcccttctgccgaagca
atgtcatctcccatacatattgtgaccacatgtctgtggtaaagttatcctgtgccagcatcaaa
atcaatgtcatctatggtctcatggttgcacttctgattggagtgtttgacatatgttgtatatc
tgtgtcctacactatgatcctccgggcagtggtcagcctgtcctctgcagatgctcggcagaagg
ccttcagcacctgcacagcccacatatctgccatcatcattacttatgttccagccttcttcacc
ttctttactcatcgttttggaggtcacaccatccctccttctcttcatatcattgtggctaatct
ttatcttcttctccctccaactctaaatcccattgtttatgggatgaagaccaaacagatcagag
atagtatcattaaattctttcacggtgaaaaaggttcaaggtga
Protein
SEQ ID NO: 20
MLISNNSYEAPC6FILNGIPGLEAVHIWISLPLCTMYIISLVGNLGLVYLIYYEESLHRPMYFFL
AMLSLIDLFTCTTTVPNALFIFWFKLKEINFTACLVQMFFVHGFTGVESGVLMLMALDRYVAICY
PLRYATILTNPVIAKAGLATFLRGVLLMIPFPFLVKRLPFCRSNVISHTYCDHMSVVKLSCASIK
INVIYGLMVALLIGVFDICCISVSYTMILRAVVSLSSADARQKAFSTCTAHISAIIITYVPAFFT
FFTHRFGGHTIPPSLHIIVANLYLLLPPTLNPIVYGMKTKQIRDSIIKFFHGEKGSR
Olfr1211
DNA
SEQ ID NO: 21
atgcaaaaccagagttttgtaacagaattcatattccttggactttcacagaaccctaaagtcca
gaaaatagtttttattgtatttttatttgtctacattgcaactgttgggggcaacatgataattg
tggtgaccattgtctgtagcccagcattgatagactgccccatgtacttctttttggcattcttg
tccctattggatgcatgcttctcttctgtcatcacaccaaagatggttgtggactccctgtatga
gaagaaaactatctcctttgaaggatgtatgatgcagttatttgctgagcacttccttgcagcag
tagaagtgattgtcttgacagccatggcctatgaccgctatgtagcaatttgcaagcccttgcac
tactcttccatcatgaactggaggctctgtggcacacttatggggatagcatggacagggggctt
cttgcattctatcatacaaattatcttcacgttgcaattgcccttctgtggaccaaatgtcatcg
atcatttcatgtgtgacttgttcccattactggaacttgcctgcactgatactcatatctttggc
cttttagtggttgccaacagtgggtctatctgcatcataatcttctctattttgctggtctccta
tggtgtcatcctgttctctctgaaagctcacagttctgaagggcgatggaaagctctctccacat
gtggatcccacattgcagttgtggttttgttctttgtcccgtgtatatttatttatgcacgtcct
ccatctgctttctcctttgataaaatggtggcgatattttatactatcctaactcccttgctcaa
tcctgtgatttatacttttcggaataaggacatgaaaaatgctatgaagaaagtgtggaagaggt
tggcagtggtttctgatggaaagtga
Protein
SEQ ID NO: 22
MQNQSFVTEFIFLGLSQNPKVQKIVFIVFLFVYIATVGCNMIIVVTIVCSPALIDCPMYFFLAFL
SLLDACFSSVITPKMVVDSLYEKKTISFEGCMVQLFAEHFLAAVEVIVLTAMAYDRYVAICKPLH
YSSIMNWRLCGTLMGIAWTGGFLHSIIQIIFTLQLPFCGPNVIDHFMCDLFPLLELACTDTHIFG
LLVVANSGSICIIIFSILLVSYGVILFSLKAHSSEGRWKALSTCGSHIAVVVLFFVPCIFIYARP
PSAFSFDKMVAIFYTILTPLLNPVIYTFRNKDMKNAMKKVWKRLAVVSDGK
OR52N5
DNA
SEQ ID NO: 23
atgcctctatttaattcattatgctggtttccaacaattcatgtgactcctccatcttttattct
taatggaatacctggtctggaaagagtacatgtatggatctccctcccactctgcacaatgtaca
tcatcttccttgtggggaatcttggtcttgtgtacctcatttattatgaggagtccttacatcat
ccgatgtattttttttttggccatgctctctccctcattgacctccttacctgcaccaccactct
acccaatgcactctgcatcttctggttcagtctcaaagaaattaacttcaatgcttgcttggccc
agatgttctttgttcatgggttcacaggtgtggagtctggggtgctcatgctcatggctctagac
cgctatgtagccatttgctaccctttgcgttatgctaccacactcaccaaccctatcattgccaa
ggctgagcttgccaccttcctgaggggtgtattgctgatgattcctttcccattcttggttaagc
gtttgcctttctgccaaagcaatattatctcccatacgtactgcgaccacatgtctgtagtaaag
ctatcttgtgccagcatcaaggtcaatgtaatctatggtctaatggttgctctcctgattggagt
gtttgacatttgttgtatatctttgtcttacactttgatcctcaaggcagcgatcagcctctctt
catcagatgctcggcagaaggctttcagcacctgcactgcccatatatctgccatcatcatcacc
tatgttccagcattcttcactttctttgcccaccgttttgggggacacacaattcccccttctct
tcacatcattgtggctaatctttatcttcttcttcccccaactctaaaccctattgtttatggag
taaagacaaaacagatacgcaagagtgtcataaagttcttccagggtgataagggtgcaggttga
Protein
SEQ ID NO: 24
MPLFNSLCWFPTIHVTPPSFILNGIPGLERVHVWISLPLCTMYIIFLVGNLGLVYLIYYEESLHH
PMYFFFGHALSLIDLLTCTTTLPNALCIFWFSLKEINFNACLAQMFFVHGFTGVESGVLMLMALD
RYVAICYPLRYATTLTNPIIAKAELATFLRGVLLMIPFPFLVKRLPFCCSNIISHTYCDHMSVVK
LSCASIKVNVIYGLMVALLIGVFDICCISLSYTLILKAAISLSSSDARQKAFSTCTAHISAIIIT
YVPAFFTFFAHRFGCHTIPPSLHIIVANLYLLLPPTLNPIVYGVKTKQIRKSVIKFFQGDKGAG
OR2L13
DNA
SEQ ID NO: 25
atggagaaatggaatcacacttcaaatgatttcattttgttgggtctgcttcccccaaatcaaac
tggaatatttctcttgtgccttatcatcctcatattctttctggcctcggtgggtaactcggcca
tgattcacctcatccacgtggatcctcgtctccacacaccgatgtactttcttctcagccagctc
tcccttatggacctgatgtacatctccaccaccgtccccaagatggcgtacaacttcctgtccgg
ccagaaaggcatctccttcctgggatgtggtgtgcaaagcttcttcttcctgaccatggcgtgtt
ctgaaggcttactcctgacctccatggcctacgaccgttatttggccatctgccactctctctat
tatcctatccgcatgagtaaaatgatgtgtgtgaagatgattggaggctcttggacactggggtc
catcaactccttggcacacacagtctttgcccttcatattccctactgcaggtctagggctattg
accatttcttctgcgatgtcccagccatgttgcttcttgcctgtacagatacttgggtctatgaa
tatatggtttttgtaagtacaagcctctttctccttttccctttcattggcatcacttcttcctg
tggccgagtcctatttgctgtctatcatatgcactcaaaggaggggagaaaaaaggccttcacca
ccatttcaacacatttaactgtagtgatcttttactatgcaccttttgtctacacctatcttcgg
cccaggaatctccgctcaccagctgaagacaagatcctggcagtcttctacaccatccttacccc
catgctcaatcccattatctacagcctgaggaataaggaagtcctgggggctatgaggagagtgt
ttgggatattctctttcctgaaagaataa
Protein
SEQ ID NO: 26
MEKWNHTSNDFILLGLLPPNQTGIFLLCLIILIFFLASVGNSAMIHLIHVDPRLHTPMYFLLSQL
SLMDLMYISTTVPKMAYNFLSGQKGISFLGCGVQSFFFLTMACSEGLLLTSMAYDRYLAICHSLY
YPIRMSKMMCVKMIGGSWTLGSINSLAHTVFALHIPYCRSRAIDHFFCDVPAMLLLACTDTWVYE
YMVFVSTSLFLLFPFIGITSSCGRVLFAVYHMHSKEGRKKAFTTISTHLTVVIFYYAPFVYTYLR
PRNLRSPAEDKILAVFYTILTPMLNPIIYSLRNKEVLGAMRRVFGIFSFLKE
OR2AJ1
DNA
SEQ ID NO: 27
atgagtgtaacagaaaatacgctcatgatcctcctcattcgcagtgactcccgactccacactcc
aatgtattttctgctcagccatctctccttaatggatatcttgcatgtttccaacatcgttccca
aaatggtcactaactttctgtcaggcagcagaactatttcatttgcaggttgtgggttccaggta
tttctgtccctcaccctcctgggtggtgagtgccttctcctggctgcaatgtcctgtgatcgcta
tgtggctatctgtcacccgctgcgctatccgattcttatgaaggagtatgccagcgctctcatgg
ctggaggctcctggctcattggggttttcaactccacagtccacacagcttatgcactgcagttt
cccttctgtggctctagggcaattgatcacttcttctgtgaagtccctgccatgttgaagttgtc
ctgtgcagacacaacacgctatgaacgaggggtttgtgtaagtgctgtgatcttcctgctgatcc
ctttctccttgatctctgcttcttatggccaaattattcttactgtcctccagatgaaatcatca
gaggcaaggaaaaagtcattttccacttgttccttccacatgattgtggtcacgatgtactatgg
gccatttatttttacatatatgagacctaaatcataccacactccagggcaggataagttcctgg
caatattctatacgatcctcacacccacactcaaccctttcatctacagctttaggaataaagat
gttctggcggtgatgaaaaatatgctcaaaagtaactttctgcacaaaaaaatgaataggaaaat
tcctgaatgtgtgttctgtctatttctatgttaa
Protein
SEQ ID NO: 28
MSVTENTLMILLIRSDSRLHTPMYFLLSHLSLMDILHVSNIVPKMVTNFLSGSRTISFAGCGFQV
FLSLTLLGGECLLLAAMSCDRYVAICHPLRYPILMKEYASALMAGGSWLIGVFNSTVHTAYALQF
PFCGSRAIDHFFCEVPAMLKLSCADTTRYERGVCVSAVIFLLIPFSLISASYGQIILTVLQMKSS
EARKKSFSTCSFHMIVVTMYYGPFIFTYMRPKSYHTPGQDKFLAIFYTILTPTLNPFIYSFRNKD
VLAVMKNMLKSNFLHKKMNRKIPECVFCLFLC
OR4C15
DNA
SEQ ID NO: 29
atgttctcaatgacaacagaagcactcaataattttgcacttggatgtaccaacttgttaatgac
tatgataccacaaattgatctgaagcaaattttcctttgtcctaattgcagactatacatgatcc
ctgttggagctttcatcttttccttgggaaacatgcaaaaccaaagctttgtaactgagtttgtc
ctcctgggactttcacagaatccaaatgttcaggaaatagtatttgttgtatttttgtttgtcta
cattgcaactgttgggggcaacatgctaattgtagtaaccattctcagcagccctgctcttctgg
tgtctcctatgtacttcttcttgggcttcctgtccttcctggatgcgtgcttctcatctgtcatc
accccaaagatgattgtagactccctctatgtgacaaaaaccatctcttttgaaggctgcatgat
gcagctctttgctgaacacttctttgctggggtggaggtgattgtcctcacagccatggcctatg
atcgttatgtggccatttgcaagcccttgcattactcttctatcatgaacaggaggctctgtggc
attctgatgggggtagcctggacagggggcctcttgcattccatgatacaaattctttttacttt
ccagcttcccttttgtggccccaatgtcatcaatcactttatgtgtgacttgtacccgttactgg
agcttgcctgcactgatactcacatctttggcctcatggtggtcatcaacagtgggtttatctgc
atcataaacttctccttgttgcttgtctcctatgctgtcatcttgctctctctgagaacacacag
ttctgaagggcgctggaaagctctctccacctgtggatctcacattgctgttgtgattttgttct
ttgtcccatgcatatttgtatatacacgacctccatctgctttttcccttgacaaaatggcggca
atattttatatcatcttaaatcccttgctcaatcctttgatttacactttcaggaataaggaagt
aaaacaggccatgaggagaatatggaacagactgatggtggtttctgatgagaaagaaaatatta
aactttaa
Protein
SEQ ID NO: 30
MFSMTTEALNNFALGCTNLLMTMIPQIDLKQIFLCPNCRLYMIPVGAFIFSLGNMQNQSFVTEFV
LLGLSQNPNVQEIVFVVFLFVYIATVGGNMLIVVTILSSPALLVSPMYFFLGFLSFLDACFSSVI
TPKMIVDSLYVTKTISFEGCMMQLFAEHFFAGVEVIVLTAMAYDRYVAICKPLHYSSIMNRRLCG
ILMGVAWTGGLLHSMIQILFTFQLPFCGPNVINHFMCDLYPLLELACTDTHIFGLMVVINSGFIC
IINFSLLLVSYAVILLSLRTHSSEGRWKALSTCGSHIAVVILFFVPCIFVYTRPPSAFSLDKMAA
IFYIILNPLLNPLIYTFRNKEVKQAMRRIWNRLMVVSDEKENIKL
OR5AC2
DNA
SEQ ID NO: 31
atggatatatcagagggaaataagactcttgtgacagagtttgttctcacaggacttacagatcg
accatggctgcacgtcctcttctttgttgtgtttttggtggtctatctcatcaccatggtgggca
accttggactgatagttctaatttggaacgacccccatcttcatatgcccatgtacttattcctt
ggtggtttagccttttcagatgcttgtacttcaacctctataacccctaggatgctggtcaattt
cttagacaagactgcaatgatatccctagctgagtgcatcacccagttttacttttttgcttcca
gtgcaactacagaatgcttcctcctggtgatgatggcctatgaccgctatgtagccatatgtaat
cccttgctttatccagtgatgatgtccaacaaactcagcgctcagttgctaagtatttcatatgt
aattggtttcctgcatcctctggttcatgtgagtttactattgcgactaactttctgcaggttta
acataatacattatttctactgtgaaattttacaactgttcaaaatttcatgcaatggtccatct
attaacgcactaatgatatttatttttggtgcttttatacaaatacccactttaatgactatcat
aatctcttatactcgtgtgctctttgatattctgaaaaaaaagtctgaaaagggcagaagcaaag
ccttctccacatgcggcgcccatctgctttctgtctcattgtactacggaactctgatcttcatg
tatgtgcgtcctgcatctggcttagctgaagaccaagacaaagtgtattctctgttttacacgat
tataattcccctgctaaacccatttatttacagcttgagaaataaaaaagtcatgcatgcattga
gaagagttataaggaagtaa
Protein
SEQ ID NO: 32
MDISEGNKTLVTEFVLTGLTDRPWLHVLFFVVFLVVYLITMVGNLGLIVLIWNDPHLHMPMYLFL
GGLAFSDACTSTSITPRMLVNFLDKTAMISLAECITQFYFFASSATTECFLLVMMAYDRYVAICN
PLLYPVMMSNKLSAQLLSISYVIGFLHPLVHVSLLLRLTFCRFNIIHYFYCEILQLFKISCNGPS
INALMIFIFGAFIQIPTLMTIIISYTRVLFDILKKKSEKGRSKAFSTCGAHLLSVSLYYGTLIFM
YVRPASGLAEDQDKVYSLFYTIIIPLLNPFIYSLRNKKVMHALRRVIRK
OR8H3
DNA
SEQ ID NO: 33
atgatgggtagaaggaatgacacaaatgtggctgacttcatccttacgggactgtcagactctga
agaggtccagatggctctgtttatgctatttctcctcatatacctaattactatgctggggaatg
tggggatgctattgataatccgcctggacctccagcttcacactcccatgtattttttccttact
cacctgtcatttattgacctcagttactcaactgtcgtcacacctaaaaccttagcgaacttact
gacttccaactatatttccttcacgggctgctttgcccagatgttctgttttgtcttcttgggta
ctgctgaatgttatcttctctcctcaatggcctatgatcgctatgcagcgatctgcagtcctcta
cactacacagttattatgcccaaaaggctctgcctcgctctcatcactgggccttatgtgattgg
ctttatggactcctttgtcaatgtggtttccatgagcagattgcatttctgtgactcaaacataa
ttcatcactttttctgtgacacttccccaattttagctctgtcctgcactgacacagacaacact
gaaatgctgatattcattatcgctggttccaccctgatggtgtcccttatcacaatatctgcatc
ctatgtgtccattctctctaccatcctgaaaattaattccacttcaggaaagcagaaagctttct
ctacttgcgtctctcatctcttgggagtcaccatcttctatggaactatgatttttacttactta
aagccaagaaagtcttattccttgggaagagatcaagtggctcctgtgttttatactattgtgat
tcccatgctgaatccactcatttatagtcttagaaacagagaagtgaaaaatgctctcattagag
tcatgcagagaagacaggactccaggtag
Protein
SEQ ID NO: 34
MVGRRNDTNVADFILTGLSDSEEVQMALFMLFLLIYLITMLGNVGMLLIIRLDLQLHTPMYFFLT
HLSFIDLSYSTVVTPKTLANLLTSNYISFTGCFAQVFCFVFLGTAECYLLSSMAYDRYAAICSPL
HYTVIMPKRLCLALITGPYVIGFMDSFVNVVSMSRLHFCDSNIIHHFFCDTSPILALSCTDTDNT
EMLIFIIAGSTLMVSLITISASYVSILSTILKINSTSGKQKAFSTCVSHLLGVTIFYGTMIFTYL
KPRKSYSLGRDQVAPVFYTIVIPMLNPLIYSLRNREVKNALIRVMQRRQDSR
OR11G2
DNA
SEQ ID NO: 35
atgaaaatcttcaacagccccagcaactccagcaccttcactggcttcatcctcctgggcttccc
ttgccccagggaggggcagatcctcctctttgtgctcttcactgttgtttacctcctgaccctca
tgggcaatggttccatcatctgtgctgtgcactgggatcagagactccacgcccccatgtacatc
ctgctcgccaacttctccttcttggagatatgttatgtcacctccacagtccccagcatgctggc
caacttcctctctgacaccaagatcatctcgttctctggctgcttcctccagttctactttttct
tctccttgggctctacagaatgctttttcctggcagttatggcatttgatcgataccttgccatc
tgtcggcctctacgctatccaaccattatgaccagacgtctctgtaccaatcttgtggtcaattg
ctgggtacttggtttcatctggttcttgattcctatcgtcaacatctcccaaatgtccttctgtg
gatctaggattattgaccacttcctatgtgacccagctcctcttctaactctcacttgcaaaaaa
ggccctgtgatagagcttgtcttttctgtcttaagtcctctgcctgtctttatgctctttctctt
cattgtggggtcctatgctctggtcgtgagagctgtgttgagggtcccttcagcagctgggagaa
gaaaggctttctccacctgtgggtctcacctggctgtggtttcactgttctacggctcagtactg
gtcatgtatgggagcccaccatctaagaatgaagctggaaagcagaagactgtgactctgtttta
ttctgttgttaccccactgcttaaccctgtgatatatagtcttaggaacaaagatatgagaaaag
ctctgaagaaattttggggaacataa
Protein
SEQ ID NO: 36
MKIFNSPSNSSTFTGFILLGFPCPREGQILLFVLFTVVYLLTLMGNGSIICAVHWDQRLHAPMYI
LLANFSFLEICYVTSTVPSMLANFLSDTKIISFSGCFLQFYFFFSLGSTECFFLAVMAFDRYLAI
CRPLRYPTIMTRRLCTNLVVNCWVLGFIWFLIPIVNISQMSFCGSRIIDHFLCDPAPLLTLTCKK
GPVIELVFSVLSPLPVFMLFLFIVGSYALVVRAVLRVPSAAGRRKAFSTCGSHLAVVSLFYGSVL
VMYGSPPSKNEAGKQKTVTLFYSVVTPLLNPVIYSLRNKDMRKALKKFWGT
OR52N2
DNA
SEQ ID NO: 37
atgtctggggacaacagctccagcctgaccccaggattctttatcttgaatggcgttcctgggct
ggaagccacacacatctggatctccctgccattctgctttatgtacatcattgctgtcgtgggga
actgtgggctcatctgcctcatcagccatgaggaggccctgcaccggcccatgtactacttcctg
gccctgctctccttcactgatgtcaccttgtgcaccaccatggtacctaatatgctgtgcatatt
ctggttcaacctcaaggagattgactttaacgcctgcctggcccagatgttttttgtccatatgc
tgacagggatggagtctggggtgctcatgctcatggccctggaccgctatgtggccatctgctac
cccttacgctatgccaccatccttaccaaccctgtcatcgccaaggctggtcttgccaccttctt
gaggaatgtgatgctcatcatcccattcactctcctcaccaagcgcctgccctattgccggggga
acttcatcccccacacctactgtgaccatatgtctgtggccaaggtatcctgtggcaatttcaag
gtcaatgctatttatggtctgatggttgctctcctgattggtgtgtttgatatctgctgtatctc
tgtatcttacactatgattttgcaggctgttatgagcctgtcatcagcagatgctcgtcacaaag
ccttcagcacctgcacatctcacatgtgttccattgtgatcacctatgttgctgcttttttcact
tttttcactcatcgttttgtaggacacaatatcccaaaccacatacacatcatcgtggccaacct
ttatctgctactgcctcctaccatgaacccaattgtttatggagtcaagaccaagcagattcagg
aaggtgtaattaaatttttacttggagacaaggttagttttacctatgacaaatga
Protein
SEQ ID NO: 38
MSGDNSSSLTPGFFILNGVPGLEATHIWISLPFCFMYIIAVVGNCGLICLISHEEALHRPMYYFL
ALLSFTDVTLCTTMVPNMLCIFWFNLKEIDFNACLAQMFFVHMLTGMESGVLMLMALDRYVAICY
PLRYATILTNPVIAKAGLATFLRNMMLIIPFTLLTKRLPYCRGNFIPHTYCDHMSVAKVSCGNFK
VNAIYGLMVALLIGVFDICCISVSYTMILQAVMSLSSADARHKAFSTCTSHMCSIVITYVAAFFT
FFTHRFVGHNIPNHIHIIVANLYLLLPPTMNPIVYGVKTKQIQEGVIKFLLGDKVSFTYDK
OR5T1
DNA
SEQ ID NO: 39
atgtttatattaataagcttcacagaagaatttgatgtgcaagtcttcctatttttattattttt
agcaatctatctattcactctaataggcaatttagggctggttgtaccgatcattggggatttct
ggcttcacagcccaatgtactattttcttggtgttttatcattcttggatgtctgctattctaca
gttgtcactccaaaaatgttggtcaatttcctggcaaaaaataaatctatttcatttcttggatg
tgcaacacagatgtttcttgcttgtacttttggaaccacagaatgctttctcttggctgcaatgg
cttatgatcgctatgtagccatctacaaccctctcctgtattcagtgagcatgtcacccagagtc
tatgtgccactcatcactgcttcctatgttgctagcattttacatgctactatacatacagtggc
tacatttagcctgtccttctgtggatccaatgaaattaggcatgtcttttgtaatatgcctcctc
tccttgctatttcttgttctgacactcacgtaatccagcttctattcttctactttgtgggctct
attgagatagtcactatcctgattgtcctgatctcctatggttttattctgttggccattctgaa
gatgcagtctgctgaagggaggagaaaagtcttctctacatgtggagctcacctaactggagtga
caatttatcatgggacaatcctcttcatgtatgtgagaccaagttccagctacacttcggacaat
gacatgatagtgtcaatattttataccattgtgattcccatgctgaatcccatcatctacagttt
gcggaacaaagatgtaaaggaggcaatcaaaagattgcttgtgagaaattggttcataaataagt
tatag
Protein
SEQ ID NO: 40
MFILISFTEEFDVQVFLFLLFLAIYLFTLIGNLGLVVPIIGDFWLHSPMYYFLGVLSFLDVCYST
VVTPKMLVNFLAKNKSISFLGCATQMFLACTFGTTECFLLAAMAYDRYVAIYNPLLYSVSMSPRV
YVPLITASYVASILHATIHTVATFSLSFCGSNEIRHVFCNMPPLLAISCSDTHVIQLLFFYFVGS
IEIVTILIVLISYGFILLAILKMQSAEGRRKVFSTCGAHLTGVTIYHGTILFMYVRPSSSYTSDN
DMIVSIFYTIVIPMLNPIIYSLRNKDVKEAIKRLLVRNWFINKL
OR4S2
DNA
SEQ ID NO: 41
atggaaaaaataaacaacgtaactgaattcattttctggggtctttctcagagcccagagattga
gaaagtttgttttgtggtgttttctttcttctacataatcattcttctgggaaatctcctcatca
tgctgacagtttgcctgagcaacctgtttaagtcacccatgtatttctttctcagcttcttgtct
tttgtggacatttgttactcttcagtcacagctcccaagatgattgttgacctgttagcaaagga
caaaaccatctcctatgtggggtgcatgttgcaactgtttggagtacatttctttggttgcactg
agatcttcatccttactgtaatggcctatgatcgttatgtggctatctgtaaacccctacattat
atgaccatcatgaaccgggagacatgcaataaaatgttattagggacgtgggtaggtgggttctt
acactccattatccaagtggctctggtagtccaactacccttttgtggacccaatgagatagatc
actacttttgtgatgttcaccctgtgttgaaacttgcctgcacagaaacatacattgttggtgtt
gttgtgacagccaacagtggtaccattgctctggggagttttgttatcttgctaatctcctacag
catcatcctagtttccctgagaaagcagtcagcagaaggcaggcgcaaagccctctccacctgtg
gctcccacattgccatggtcgttatctttttcggcccctgtacttttatgtacatgcgccctgat
acgaccttttcagaggataagatggtggctgtattttacaccattatcactcccatgttaaatcc
tctgatttatacactgagaaatgcagaagtaaagaatgcaatgaagaaactgtggggcagaaatg
ttttcttggaggctaaagggaaatag
Protein
SEQ ID NO: 42
MEKINNVTEFIFWGLSQSPEIEKVCFVVFSFFYIIILLGNLLIMLTVCLSNLFKSPMYFFLSFLS
FVDICYSSVTAPKMIVDLLAKDKTISYVGCMLQLFGVHFFGCTEIFILTVMAYDRYVAICKPLHY
MTIMNRETCNKMLLGTWVGGFLHSIIQVALVVQLPFCCPNEIDHYFCDVHPVLKLACTETYIVGV
VVTANSGTIALGSFVILLISYSIILVSLRKQSAEGRRKALSTCGSHIAMVVIFFGPCTFMYMRPD
TTFSEDKMVAVFYTIITPMLNPLIYTLRNAEVKNAMKKLWGRNVFLEAKGK
FLAG ® tag
DNA
SEQ ID NO: 43
gattacaaggacgacgacgataag
Protein
SEQ ID NO: 44
DYKDDDDK
Rho tag
DNA
SEQ ID NO: 45
atgaacgggaccgagggcccaaacttctacgtgcctttctccaacaagacgggcgtggtg
Protein
SEQ ID NO: 46
MNGTEGPNFYVPFSNKTGVV
Lucy tag
DNA
SEQ ID NO: 47
atgagaccccagatcctgctgctcctggccctgctgaccctaggcctggct
Protein
SEQ ID NO: 48
MRPQILLLLALLTLGLA
Human G-protein alpha subunit G_{olf}
DNA
SEQ ID NO: 49
atgggtctgtgctacagtctgcggccgctgcttttcgggggcccaggggacgacccctgcgcggc
ctcggagccgccggtggaggacgcgcagcccgccccggccccggccctggccccagtccgggcgg
ccgcaagggacacggcccggaccctgctccctcggggcggcgaagggagcccggcatgcgctcgg
cccaaagcagacaagccgaaggagaagcggcagcgcaccgagcagcttagtgccgaggagcgcga
ggcggccaaggagcgcgaggcggtcaaggaggcgaggaaagtgagccggggcatcgaccgcatgc
tgcgcgaccagaagcgcgacctgcagcagacgcaccggctcctgctgctcggggctggtgagtct
gggaaaagcactatcgtcaaacagatgaggatcctgcacgtcaatgggtttaatcccgaggaaaa
gaaacagaaaattctggacatccggaaaaatgttaaagatgctatcgtgacaattgtttcagcaa
tgagtactataatacctccagttccgctggccaaccctgaaaaccaatttcgatcagactacatc
aagagcatagcccctatcactgactttgaatattcccaggaattctttgaccatgtgaaaaaact
ttgggacgatgaaggcgtgaaggcatgctttgagagatccaacgaataccagctgattgactgtg
cacaatacttcctggaaagaatcgacagcgtcagcttggttgactacacacccacagaccaggac
ctcctcagatgcagagttctgacatctgggatttttgagaacgattccaagtggacaaagtaaac
ttccacatgtttgatgttggtggccagagggatgagaggagaaaatggatccagtgctttaacga
tgtcacagctatcatttacgtcgcagcctgcagtagctacaacatggtgattcgagaagataaca
acaccaacaggctgagagagtccctggatctttttgaaagcatctggaacaacaggtggttacgg
accatttctatcatcttgttcttgaacaaacaagatatgctggcagaaaaagtcttggcagggaa
atcaaaaattgaagactatttcccagaatatgcaaattatactgttcctgaagacgcaacaccag
atgcaggagaagatcccaaagttacaagagccaagttctttatccgggacctgtttttgaggatc
agcacggccaccggtgacggcaaacattactgctacccgcacttcacctgcgccgtggacacaga
gaacatccgcagggtgttcaacgactgccgcgacatcatccagcggatgcacctcaagcagtatg
agctcttgtga
Protein
SEQ ID NO: 50
MGLCYSLRPLLFGGPGDDPCAASEPPVEDAQPAPAPALAPVRAAARDTARTLLPRGGEGSPACAR
PKADKPKEKRQRTEQLSAEEREAAKEREAVKEARKVSRGIDRMLRDQKRDLQQTHRLLLLGAGES
GKSTIVKQMRILHVNGFNPEEKKQKILDIRKNVKDAIVTIVSAMSTIIPPVPLANPENQFRSDYI
KSIAPITDFEYSQEFFDHVKKLWDDEGVKACFERSNEYQLIDCAQYFLERIDSVSLVDYTPTDQD
LLRCRVLTSGIFETRFQVDKVNFHMFDVGGQRDERRKWIQCFNDVTAIIYVAACSSYNMVIREDN
NTNRLRESLDLFESIWNNRWLRTISIILFLNKCDMLAEKVLAGKSKIEDYFPEYANYTVPEDATP
DAGEDPKVTRAKFFIRDLFLRISTATGDGKHYCYPHFTCAVDTENIRRVFNDCRDIIQRMHLKQY
ELL
Human G-protein alpha subunit Gα15
DNA
SEQ ID NO: 51
atggcccggtcgctgacctggcgctgctgcccctggtgcctgacggaggatgagaaggccgccgc
ccgggtggaccaggagatcaacaggatcctcttggagcagaagaagcaggaccgcggggagctga
agctgctgcttttgggcccaggcgagagcgggaagagcaccttcatcaagcagatgcggatcatc
cacggcgccggctactcggaggaggagcgcaagggcttccggcccctggtctaccagaacatctt
cgtgtccatgcgggccatgatcgaggccatggagcggctgcagattccattcagcaggcccgaga
gcaagcaccacgccagcctggtcatgagccaggacccctataaagtgaccacgtttgagaagcgc
tacgctgcggccatgcagtggctgtggagggatgccggcatccgggcctgctatgagcgtcggcg
ggaattccacctgctcgattcagccgtgtactacctgtcccacctggagcgcatcaccgaggagg
gctacgtccccacagctcaggacgtgctccgcagccgcatgcccaccactggcatcaacgagtac
tgcttctccgtgcagaaaaccaacctgcggatcgtggacgtcgggggccagaagtcagagcgtaa
gaaatggatccattgtttcgagaacgtgatcgccctcatctacctggcctcactgagtgaatacg
accagtgcctggaggagaacaaccaggagaaccgcatgaaggagagcctcgcattgtttgggact
atcctggaactaccctggttcaaaagcacatccgtcatcctctttctcaacaaaaccgacatcct
ggaggagaaaatccccacctcccacctggctacctatttccccagtttccagggccctaagcagg
atgctgaggcagccaagaggttcatcctggacatgtacacgaggatgtacaccgggtgcgtggac
ggccccgagggcagcaagaagggcgcacgatcccgacgcctcttcagccactacacatgtgccac
agacacacagaacatccgcaaggtcttcaaggacgtgcgggactcggtgctcgcccgctacctgg
acgagatcaacctgctgtga
Protein
SEQ ID NO: 52
MARSLTWRCCPWCLTEDEKAAARVDQEINRILLEQKKQDRGELKLLLLGPGESCKSTFIKQMRII
HGAGYSEEERKGFRPLVYQNIFVSMRAMIEAMERLQIPFSRPESKHHASLVMSQDPYKVTTFEKR
YAAAMQWLWRDAGIRACYERRREFHLLDSAVYYLSHLERITEEGYVPTAQDVLRSRMPTTGINEY
CFSVQKTNLRIVDVGGQKSERKKWIHCFENVIALIYLASLSEYDQCLEENNQENRMKESLALFGT
ILELPWFKSTSVILFLNKTDILEEKIPTSHLATYFPSFQGPKQDAEAAKRFILDMYTRMYTGCVD
GPEGSKKGARSRRLFSHYTCATDTQNIRKVFKDVRDSVLARYLDEINLL
Motif
SEQ ID NO: 53
MAYDRYVAIC
Motif
SEQ ID NO: 54
FSTCSSH
Motif
SEQ ID NO: 55
PMLNPFIY

The following examples are illustrative only and are not meant to limit the scope of invention as set forth in the Summary, Description or in the Claims.

### EXAMPLES

### Example 1

Identification of novel mouse and human DMTS activated odorant receptors. The identification of new odorant receptors was performed according to the method disclosed in WO2014/210585. Briefly, murine olfactory sensory neurons were exposed to DMTS and screened using a Ca²⁺ imaging technique. Neurons that were activated by DMTS were further isolated for full transcriptome analysis to identify the responsive odorant receptor. The cDNA corresponding to the isolated cell mRNA was generated and amplified by PCR based method (Clontech/Takara, SMARTer® Ultra® Low Input RNA Kit for Sequencing—v3, cat. 634848). Amplified cDNA was then used to generate an Illumina cDNA library for Next-Generation-Sequencing and the generation of 100 base pair single read sequences. Sequences were aligned to a mouse reference genome (such as UCSC version mm10) in order to generate the full transcriptome. Because only a single odorant receptor (OR) is strongly transcribed per olfactory sensory neuron, the subsequent identification of the DMTS responsive OR can be achieved. Phylogenetic relationship evaluation using sequence similarity searches were then used to identify the corresponding human OR. Similar functional response profiles between orthologous OR pairs are often observed [e.g. Adipietro, K. A, et al. PLoS Genet. 8, e1002821-e1002821 (2012), Sato-Akuhara, N. et al. J. Neurosci. 36, 4482-4491 (2016) and WO2016/201152] and can be used for human OR identification [e.g. Armelin-Correa L. M. and Malnic B. J Agric Food Chem. doi: 10.1021/acs.jafc.6b04998 (2017)]. FIG. 1 shows the pairwise identity levels between the receptors mentioned herein. ORs that do not share an orthologous and/or paralogous relationship only share an average of 39%±13.1%. All orthologous and paralogous relationship share an average of 77%±7.5%. All paralogs and orthologs pairs are indicated by a gray shaded cell. They represent the highest identity levels as supported by the amino acid identity levels indicated.

### Example 2

Functional Characterization of Mouse and Human DMTS Receptors

Functional dose-response experiments were performed to evaluate the level of DMTS activity of the modified cell line expressing individual putative DMTS receptors. Using a cell-based assay, mouse receptor Olfr1193, Olfr1093, Olfr1097, Olrf166, Olfr169, Olfr738 and Olfr742, and human receptor OR4S2 and OR52N5 were tested in an HEK293T cell line wherein the endogenous RTP1 gene has been activated and the odorant receptor chaperone protein was expressed (described in WO2016/201153). The mouse receptor genes were tagged with a Lucy-FLAG®-Rho tag combination and the human receptors were tagged with a Rho-FLAG® tag combination resulting in a tag::receptor fusion protein for the cell based assay. Receptor genes were co-transfected with the canonical olfactory human G-protein alpha subunit Golf gene and were exposed to increasing concentrations of the malodor odorant. Co-expression of the human Golf activates the Gs transduction pathway that leads to an internal cAMP increase upon binding to the appropriate ligand. Odorant-induced activity was detected by measuring the cAMP increase in the cytosol using an Homogeneous Time Resolved Fluorescence (HTRF) based kit (CisBio, cAMP dynamic 2 kit, cat. 62AM4PEJ). A dose-dependent increase of the receptor activity is observed specifically for DMTS but not for butyric acid, another known malodor used as a negative control (FIG. 1). The activity levels are reported by the potency of the DMTS induced-response for each receptor as a measure of the EC50 value (the effective concentration at which the receptor responds at its half-maximal activation efficacy level for a given compound).

### Example 3

Functional Characterization of Additional Human DMTS Receptors

Functional dose-response experiments were performed to evaluate the level of DMTS activity of the modified cell lines expressing individual putative DMTS receptors. A cell line stably expressing a human receptor was generated in a HEK293T cell line for each of the following human receptors: OR2L13, OR4C15, OR5AC2, OR8H3, OR11G2 or OR52N2. The receptors were tagged with a FLAG®-Rho tag combination and stably co-expressed with the human G-protein alpha subunit Gals for the cell based assay. Co-expression of the human Gals activates the Gq transduction pathway that leads to an internal Ca²⁺ increase upon binding to the appropriate ligand. Receptors were exposed to increasing concentrations of DMTS. Odorant-induced activity was detected by measuring the Ca²⁺ increase in the cytosol using a calcium sensitive fluorescent dye (Molecular Devices, Calcium 5 dye, cat. R8186) and measuring the change in Relative Fluorescence Ratio (RFU) using a fluorometric imaging plate reader (Molecular Devices, FLIPR) following odorant exposure. A dose-dependent increase of receptor activity was recorded and a corresponding dose-response curve is shown for DMTS (FIG. 2). A cell line lacking a receptor was used as a control for non-specific activity at high compound concentrations (‘No receptor’). The activity levels are reported by the potency of the DMTS induced-response for each receptor as a measure of the EC50 value.

### Example 4

Identification of DMTS Receptor Inhibitors

The stable cell lines described in Example 3 were used as an antagonist screening platform to identify compounds that have the property to decrease the DMTS induced receptor activity. Each stable cell line expressing a human odorant receptor was screened with a volatile compound library for their inhibitory properties and potential DMTS smell inhibition. First, individual binary mixtures of DMTS with each one of the test compounds were presented to the cells. Single point monitoring of the DMTS induced cell activity in the presence or absence of a test compound allowed for the identification of compounds with a putative suppression or inhibitory effect. These hits were further confirmed in an inhibitory dose-response curve assay to evaluate the potency of activity inhibition as a measure of the IC50 (the inhibitor concentration at which the receptor activity is inhibited by the half-maximal inhibition efficacy level of a given test compound). A dose-dependent decrease of receptor activity was recorded with increasing concentrations of test compounds in the presence of a single activating concentration of DMTS (EC80) and corresponding dose-response inhibition curves were obtained. The compounds in the following table are examples of compounds that decreased the DMTS induced activity of at least one receptor as depicted in FIG. 4.

| | |
|---|---|
| Cyclemone A | 1,2,3,4,5,6,7,8-octahydro-8,8-dimethyl-2- |
| | naphthalenecarbaldehyde (A) + (B,C,D) + octahydro- |
| | 5,5-dimethyl-2-naphthalenecarbaldehyde |
| Geonol | (+-)-perhydro-4alpha,8abeta-dimethyl-4a-naphthalenol |
| Hivemal | 3-(3,3-dimethyl-2,3-dihydro-1H-inden-5-yl)propanal |
| Neo ® | (A) + 3-(1,1-dimethyl-2,3-dihydro-1H-inden-4- |
| | yl)propanal (B) + 3-(1,1-dimethyl-2,3-dihydro-1H- |
| | inden-5-yl)propanal (C) |
| Lilyflore ® | (+-)-2,5-dimethyl-2-indanmethanol |
| Patchouli | (-)- (3R,6S ,8S)-2,2,6,8- |
| alcohol | tetramethyltricyclo[5.3.1.0~3,8~]undecan-3-ol |
| Patchouli Oil | patchouli oil |
| Rosinol Cryst | (+-)-2,2,2-trichloro-1-phenylethyl acetate |
| Spiranol ® | 5RS,6RS)-2,6,10,10-tetramethyl-1-oxaspiro[4.5]decan- |
| | 6-ol |
| Terranol | 2,2,7,7-tetramethyltricyclo[6.2.1.0~1,6~]undecan-6-ol |
| Wolfwood ® | (+)-(1S,2S,3S,5R)-2,6,6- |
| | trimethylspiro[bicyclo[3.1.1]heptane-3,1′-cyclohexane]- |
| | 2′-en-4′-one |

In FIG. 4, the length of the bars indicate the potency of activity inhibition (IC50, expressed in negative log molar concentration) of the select compounds for each receptor. The absence of a bar indicates absence of inhibition for the corresponding receptor compound pair. These compounds can specifically bind and block the activity of DMTS or DMDS receptors, and thus may suppress or inhibit the smell of DMTS or DMDS. Such compounds may therefore be used as malodour counteractants for latrine or oral odor control applications.

### Example 5

Response Profiles of Mouse and Human DMTS Receptors to DMDS

Functional dose-response experiments were performed to confirm the DMTS activity on the DMTS receptors identified in Example 2 and to further characterize their response to DMDS. Using the same cell-based assay described in Example 2, mouse receptors Olfr1193, Olfr1093, Olfr1097, Olfr166, Olfr169, Olfr738, Olfr742 and of human odorant receptor OR4S2 were tested with increasing concentration of DMTS, DMDS or butyric acid (control). A strong dose-dependent increase of the receptor activity is observed for DMTS and DMDS relative to the butyric acid control compound. A weak response for OR4S2 and Olfr169 to butyric acid was visible but was too weak to consider butyric acid as a representative ligand for these receptors. The activity levels are reported by the potency of DMTS or DMDS induced-response for each receptor as a measure of the EC50 value, EC50_{DMTS} and EC50_{DMDS} respectively. A mock transfection control experiment in which the cells do not express the odorant receptors did not show activity upon DMTS or DMDS exposure. Competitive antagonists at the malodor binding site of these receptors have the potential to reduce the unpleasant perception of both DMTS and DMDS.

1. A method for identifying a compound that binds, suppresses, blocks, inhibits, and/or modulates the activity of an olfactory receptor that is activated by a malodor-causing substance comprising:
a. contacting a test substance and a malodor-causing substance to at least one olfactory receptor selected from the group consisting of Olfr1193, Olfr1093, Olfr166, Olfr169, Olfr738, Olfr742, Olfr207, Olfr665, Olfr669, Olfr1211, OR52N5, OR2L13, OR2AJ1, OR4C15, OR5AC2, OR11G2, OR52N2, and OR5T1;
b. measuring the response of the olfactory receptor to the malodor-causing substance by measuring the response of the olfactory receptor in the presence and absence of the test sub stance;
c. identifying a test substance that modulates the response of the olfactory receptor on the basis of the response that was measured in the presence and absence of the test substance; and
d. selecting the identified test substance as a compound that modulates the response of the olfactory receptor to the malodor-causing substance,
wherein the malodor-causing substance is dimethyl trisulfide (DMTS) or dimethyl disulfide (DMDS).

2. (canceled)

3. A method for identifying a compound that binds, suppresses, blocks, inhibits, and/or modulates the activity of at least one olfactory receptor that is activated by a malodor-causing substance, the method comprising:
a. contacting the receptor, or a chimera or fragment thereof with a compound; and
b. determining whether the compound has an effect on the activity of the receptor;
wherein the receptor is a polypeptide
i) comprising an amino acid sequence having at least 70%, 75%, 80%, 85%, 90%, 95%, 96%, 97%, 98%, 99%, or 100% sequence identity to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 36, SEQ ID NO: 38, or SEQ ID NO: 40; or
ii) encoded by a nucleic acid molecule comprising a nucleic acid sequence having at least 70%, 75%, 80%, 85%, 90%, 95%, 96%, 97%, 98%, 99%, or 100% sequence identity to SEQ ID NO: 1, SEQ ID NO: 3, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 39, SEQ ID NO: 31, SEQ ID NO: 35, SEQ ID NO: 37, SEQ ID NO: 39 or the reverse complement thereof;
wherein the malodor-causing substance is dimethyl trisulfide (DMTS) or dimethyl disulfide (DMDS).

4. A method of identifying a compound that putatively modulates DMTS or DMDS associated malodor comprising: (i) contacting a cell line that expresses a DMTS or DMDS receptor polypeptide with at least one compound; (ii) screening for compounds that bind, suppress, block, inhibit, and/or modulate the activity of said olfactory receptor polypeptide; and (iii) identifying a compound that putatively modulates DMTS or DMDS associated malodor if it binds, suppresses, blocks, inhibits, and/or modulates the activity of said DMTS or DMDS receptor polypeptide,
wherein the receptor polypeptide
a. comprises an amino acid sequence having at least 90% sequence identity to SEQ ID NO: 2, SEQ ID NO: 4, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 36, SEQ ID NO: 38, or SEQ ID NO: 40; or
b. is encoded by a nucleic acid molecule comprising a nucleic acid sequence having at least 70%, 75%, 80%, 85%, 90%, 95%, 96%, 97%, 98%, 99%, or 100% sequence identity to SEQ ID NO: 1, SEQ ID NO: 3, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 39, SEQ ID NO: 31, SEQ ID NO: 33, SEQ ID NO: 35, SEQ ID NO: 37, SEQ ID NO: 39 or the reverse complement thereof.

5.-15. (canceled)


SEQUENCE LISTING (55 sequences)

SEQ ID NO: 1 - DNA, 957 bp, Mus musculus
  source 1..957: mol_type = genomic DNA
atggcctcaaggacttattccatggaagaagtaaataatgtcactgaattcattttcttgggtctttctcagaaccctgaggttgaaaaagtgtgctttgtggtgttctccttcttttacatggtcattctgctaggaaacctcctcatcatgttgacagtttgcagtggcaatcttttcaagtttcccatgtattttttcctcaactttctgtcttttgtggacatttgctactcctcagtcacagcacccaagatgattattgacctgttagtgaagaaaaagactatatcctatgtggggtgcatgttacaactctttgtggttcatttctttggttgcactgagatcttcattcttactgtcatggcctatgatagatatgtggccatttgtaaacctctccactatatgactatgatggaccgggaaagatgcaataagatgttgctcggaacatggatcggtggcttcttacattctattatccaagtggctcttgtggtccagctccccttttgtggaccgaatgagattgatcactatttctgtgatgtacatcctgtactgaaacttgcctgcactgacacttacattgttggtatttttgtgacagcaaacagtggcaccattgcattgggaagttttgtcatcttgctgatctcatacacagtcattctcatgtctctgagaaagcagtcatctgaaggcagacgcaaagctctctccacttgtggatcccacattgctgttgtcatcattttttttggcccctgtacttttatgtatatgcggcctgacactaccttctctgaggacaagatggtagctatattttacaccattatcactcccatgctgaatcctctaatttacactctaagaaatgcagaagtaaagaatgcaatgagaaaactgtgggctagaaagttttcctgggaaactactgggaaatag

SEQ ID NO: 2 - protein, 318 aa, Mus musculus
MASRTYSMEEVNNVTEFIFLGLSQNPEVEKVCFVVFSFFYMVILLGNLLIMLTVCSGNLFKFPMYFFLNFLSFVDICYSSVTAPKMIIDLLVKKKTISYVGCMLQLFVVHFFGCTEIFILTVMAYDRYVAICKPLHYMTMMDRERCNKMLLGTWIGGFLHSIIQVALVVQLPFCGPNEIDHYFCDVHPVLKLACTDTYIVGIFVTANSGTIALGSFVILLISYTVILMSLRKQSSEGRRKALSTCGSHIAVVIIFFGPCTFMYMRPDTTFSEDKMVAIFYTIITPMLNPLIYTLRNAEVKNAMRKLWARKFSWETTGK

SEQ ID NO: 3 - DNA, 969 bp, Mus musculus
  source 1..969: mol_type = genomic DNA
atggaaaagatcacatcagctgtggatgtccacaatattccattaaagaacatgactgaagccaccatgtttattctcttaggattcacagatgactttgaactccaagtcttcctgtttttactgtttcttgctatttatctcttcactctggtaggaaactttggactggttgttttggtcattggggattgtcggctacacaaccccatgtactatttcctaagtgttttgtctttcctggatgcttgctattctacagttgttacacccaaaatgttggtcaactttctaagtgaaaataagtccatttcattccttgcatgtgcaacccaaatgcttctctttgtttcgttgggaaccacagaatgctttctcctggcagcaatggcttatgaccgatatgtagccatctacaacccacttctgtatacagtggccatgtcacccagagtatacctgccactcatcattgcttcctatgctggtggagttgtgcatggtgctatccacacagtggccactttcagtctgtccttctgtggatccaatgaaattaagcatgtcttctgtgacatccctgcattgcttgctctttcttgttctgatacccacacaaatgagcttctagtcttgtacttggtgggcttgattgagattgttaccatcctgattgttctggtctcctatggattcatcctctttgccattctgaacatgcattctgctgagggtaggaggaaagtgttctctacatgtggctctcacctcactggagtctctatttaccatggtacaatccttttcacttatatgaggcctagttccagttatgcttcaaatcatgacatggtagtgtcaatattttacaccattgtgatacccatgttgaatcctatcatctatagtttgaggaacaaagatgtaaaagtagcatttaataaattgtggagaaaatgtgattcataa

SEQ ID NO: 4 - protein, 322 aa, Mus musculus
MEKITSAVDVHNIPLKNMTEATMFILLGFTDDFELQVFLFLLFLAIYLFTLVGNFGLVVLVIGDCRLHNPMYYFLSVLSFLDACYSTVVTPKMLVNFLSENKSISFLACATQMLLFVSLGTTECFLLAAMAYDRYVAIYNPLLYTVAMSPRVYLPLIIASYAGGVVHGAIHTVATFSLSFCGSNEIKHVFCDIPALLALSCSDTHTNELLVLYLVGLIEIVTILIVLVSYGFILFAILNMHSAEGRRKVFSTCGSHLTGVSIYHGTILFTYMRPSSSYASNHDMVVSIFYTIVIPMLNPIIYSLRNKDVKVAFNKLWRKCDS

SEQ ID NO: 5 - DNA, 948 bp, Mus musculus
  source 1..948: mol_type = genomic DNA
atgagtgcctgtaatcatacaaatgaacctgagttcacgcttgtgggactgacagactccaaggagattcagctggtcctctctgttttgtttctcctgatatacatgctcactgtcttgggaaacataggtatgatactgatcattcatctagatgtccagctccacactccaatgtattttttcctcacccacttgtcattccttgacctcagttactcaactgtaatcacacctaaaaccttacagaatacgctgacctccataaaaaatatttccttcatgggatgcttcacccagttgtatttctttgtcctcttggcagcttctgaatgttttatactttcgtcaatggcctatgaccgctatgtagctatctgcaaccctctacactatccagttattatgtcccctaggcgctcatatactctcatcactgtgtcctacatgattggagttttggattcttctgtcactgtcttttgcttaagcacactggatttctgcaactccaaagtaattcatcacttcttttgtgacacattcccaattttagctctgtcctgcagtgatacctataatgcagaagccactatattcgttttagctggttccactctattgctgtcgctcatcacgatatcctcatcctatgtatctattctctctacaattttgaagataaattcttcttcaggaaagcacaaagccttctctacatgtgcctcacatcttataggagtcactgttttttatggtacaatgatctttacttatttaaaaccaagtacgtcctactccctgggaaaggatcaagtagcctctgttttttatactatagtgattcccatgctgaacccacttatctatagtctcaggaacaaagaagtgaaaagtgctgttgttagagttatgaagaagagagagtgcatccagaaactagaataa

SEQ ID NO: 6 - protein, 315 aa, Mus musculus
MSACNHTNEPEFTLVGLTDSKEIQLVLSVLFLLIYMLTVLGNIGMILIIHLDVQLHTPMYFFLTHLSFLDLSYSTVITPKTLQNTLTSIKNISFMGCFTQLYFFVLLAASECFILSSMAYDRYVAICNPLHYPVIMSPRRSYTLITVSYMIGVLDSSVTVFCLSTLDFCNSKVIHHFFCDTFPILALSCSDTYNAEATIFVLAGSTLLLSLITISSSYVSILSTILKINSSSGKHKAFSTCASHLIGVTVFYGTMIFTYLKPSTSYSLGKDQVASVFYTIVIPMLNPLIYSLRNKEVKSAVVRVMKKRECIQKLE

SEQ ID NO: 7 - DNA, 939 bp, Mus musculus
  source 1..939: mol_type = genomic DNA
atggagaaatggaatcagagctcaagtgattttactctgttaggactgcttccacaaaaccaaacaggcctgctacttttgatgctcatcatctttgtcttctctctggctttgtgtggcaactcaggaatgatccacctcattcgtgtggatccaaggctccacacccccatgtactttctcctcagtcagctctctctcatggacctgatgtacatttctaccactgttcccaagatggcatttaacttcctttctggccagaaaagcatctcttttctgggctgtggagtgcaatccttcttcttcctgactatggcatgttctgagggcttgctcttggcttccatggcttatgatcgttttgtggctatctgccatccccttcactatcccattcgcatgagcaaaataatgtgtctgaagatgatcataggatcctggatattgggctcaatcaactctttagcacataccgtctatgcccttcatattccttactgccattctaggtccattaaccatttcttctgtgatgttccagccatgttgcccctggcctgtatggacacttgggtttatgagtacatggtgtttgtgagcacaagcctgtttctcctactgcctttccttggtatcacagcttcctatggtcgggtcctttttgctgtcttccacatgcgctcaaaagagggaaagaagaaggccttcaccacatgctcaactcacttaactgtggtgacattttactatgcaccttttgtctatacctatcttcgacctaggagtcttcgctccccaacagaagataagattctggctgttttctacactatccttacccccatgctcaaccccatcatttatagtctgaggaataaggaggtcctgggggccatgacaagagtccttggtacttttccttcaactaaaccgtaa

SEQ ID NO: 8 - protein, 312 aa, Mus musculus
MEKWNQSSSDFTLLGLLPQNQTGLLLLMLIIFVFSLALCGNSGMIHLIRVDPRLHTPMYFLLSQLSLMDLMYISTTVPKMAFNFLSGQKSISFLGCGVQSFFFLTMACSEGLLLASMAYDRFVAICHPLHYPIRMSKIMCLKMIIGSWILGSINSLAHTVYALHIPYCHSRSINHFFCDVPAMLPLACMDTWVYEYMVFVSTSLFLLLPFLGITASYGRVLFAVFHMRSKEGKKKAFTTCSTHLTVVTFYYAPFVYTYLRPRSLRSPTEDKILAVFYTILTPMLNPIIYSLRNKEVLGAMTRVLGTFPSTKP

SEQ ID NO: 9 - DNA, 942 bp, Mus musculus
  source 1..942: mol_type = genomic DNA
atggaatatgagaactacacttttaacagcgacttcatcctcttgggactgttctcttcttcaaagacaagcttaacttttttctcatttatatttttcatttttattatggctataacagaaaatgccctcatgatcctcctaatccacagggattctcgactccataccccaatgtatttcctgcttagtcatctctccttcatggatatcttgcacatttccaacattgttcctaaaatgattgctgacttcctctcaggcagcagaactatttcctttgcaggctgtgccttccagatatttctctctcttaccttgctaggtggtgagtgccttctcctggcagccatgtcctatgatcgatatgtggccatctgccacccacttcgctaccctgtgctgatgagggataactccagtaggctcctggctgcaggctcctggctggtggggatcctcaactccatagtacacacagtttttgcactccactttcccttctgccactcaagagccattgatcactttttctgcgaagtccctgccatgttgaaattgtcatgtatagacacaacacactatgaacgaggcgtttatgtgagtggcattatttttctgctgatcccattttccatgatctctatatcttatgtgcaaattctcctcactgtattccaaatgcagtcatcaggggcccggcaaaagtccttttccacctgttccttccacatggttgttgtcataatgtactatgggccattcatttttacatatatgagacctcgctcataccacactccagggcaggataaatttttggcaatattctacaccatcctgacacccacactcaaccccataatctacagctttcgtaataaagatgtccttatggctgtgaaaaacatcgtccaaagtaattttttgaataaaaaatga

SEQ ID NO: 10 - protein, 313 aa, Mus musculus
MEYENYTFNSDFILLGLFSSSKTSLTFFSFIFFIFIMAITENALMILLIHRDSRLHTPMYFLLSHLSFMDILHISNIVPKMIADFLSGSRTISFAGCAFQIFLSLTLLGGECLLLAAMSYDRYVAICHPLRYPVLMRDNSSRLLAAGSWLVGILNSIVHTVFALHFPFCHSRAIDHFFCEVPAMLKLSCIDTTHYERGVYVSGIIFLLIPFSMISISYVQILLTVFQMQSSGARQKSFSTCSFHMVVVIMYYGPFIFTYMRPRSYHTPGQDKFLAIFYTILTPTLNPIIYSFRNKDVLMAVKNIVQSNFLNKK

SEQ ID NO: 11 - DNA, 936 bp, Mus musculus
  source 1..936: mol_type = genomic DNA
atgaaagcctttagcagccccagcaactccagcatcatcactggcttcatcctcctgggcttcccctgccccaaggaggggcaaatcctcctctttgtgctcttcttcattatctacatccttaccctcatgggcaatgcttccatcatatgtgctgtgtgctatgataagaaacttcacagccccatgtacctcctgctggccaacttctccttcctagaaatctggtatgtcacctccacagtccccaacatgttggccaacttcctctctgacacgaaggtcatctctttctctggatgcttcctgcagttctatttcttcttctccttgggttctacagaatgctttttcctggcagtcatggcatttgatcgataccttgccatctgcagacctctacattatccttctctcatgactgggcgcctctgcaacatccttgtgatcagttgctgggtgcttggtttcctctggttccctgttcccatcatcatcatctcccaaatgtccttctgtggatccagaattatagaccacttcctgtgtgacccaggccctctgttggccctcacctgtgtgagaaattctttaattgagatgactagctctactttaagttccctgcttttatttgttccatttttttttatcatggggtcttatgctctagtaatgagggctgtgctcagggtcccttcagcagctggacgaagaaaggccttctccacctgtgggtcacacttgactgtggtttctcttttctatggctcagtgatggtcatgtatgtgagcccaacatctgaacatgcagctggagtgcaaaaacttgtgactctgttttattctgtggttactcccctccttaatcctgtgatatacagtctgaggaacagagatatgaaacatgcaatgaaaaagttactgaaaatgtaa

SEQ ID NO: 12 - protein, 311 aa, Mus musculus
MKAFSSPSNSSIITGFILLGFPCPKEGQILLFVLFFIIYILTLMGNASIICAVCYDKKLHSPMYLLLANFSFLEIWYVTSTVPNMLANFLSDTKVISFSGCFLQFYFFFSLGSTECFFLAVMAFDRYLAICRPLHYPSLMTGRLCNILVISCWVLGFLWFPVPIIIISQMSFCGSRIIDHFLCDPGPLLALTCVRNSLIEMTSSTLSSLLLFVPFFFIMGSYALVMRAVLRVPSAAGRRKAFSTCGSHLTVVSLFYGSVMVMYVSPTSEHAAGVQKLVTLFYSVVTPLLNPVIYSLRNRDMKHAMKKLLKM

SEQ ID NO: 13 - DNA, 936 bp, Mus musculus
  source 1..936: mol_type = genomic DNA
atgaaaaccctcagcagccccagcaactccagcaccatcactggcttcatcctcttgggcttcccctgccccagggaggggcaaatcctcctctttgtgaccttcttcattgtttacatactcattcttatgggcaatgcttccatcatctgtgctgtgtactgtgatcagagcctccacacccccatgtacttcctgctggccaacttctccttcctggagatctggtatgtcacctccacagtccccaacatgttggccaacttcctttcagacaccaaggtcatctctttctctggatgcttcctgcagttctatttcttcttctcctttggttctacagaatgctttttcctggcagtcatggcatttgatcgataccttgccatctgtaggccactacattatccttctctcatgactgggcacctctgcaacatccttgtgatcagttgctgggtgcttggtttcctctggttccctgtacccatcatcatcatctcccagatgtccttctgtgggtccagaattatagaccacttcctgtgtgacccaggccctcttttggcccttgcctgttccagagccccattgatggaggttttctggacaattataatgtctatgctcctggttattcctttcctcttcatcatgggaacttacatattggtcctaagagctgtgtttagacttccttcaagagatggacaaaaaaaggccttctccacttgcgggtctcatctcacagtagtttcactcttttattgctcagtgatgaaaatgtatttgagcccaacatctgagcatgaagctggaatgcagaagcttgtaactctattttattctgtgggtactccactacttaatcctgtgatatacagtctgaggaacaaagatatgaaaaatgccctgcagaagattttaagaacataa

SEQ ID NO: 14 - protein, 311 aa, Mus musculus
MKTLSSPSNSSTITGFILLGFPCPREGQILLFVTFFIVYILILMGNASIICAVYCDQSLHTPMYFLLANFSFLEIWYVTSTVPNMLANFLSDTKVISFSGCFLQFYFFFSFGSTECFFLAVMAFDRYLAICRPLHYPSLMTGHLCNILVISCWVLGFLWFPVPIIIISQMSFCGSRIIDHFLCDPGPLLALACSRAPLMEVFWTIIMSMLLVIPFLFIMGTYILVLRAVFRLPSRDGQKKAFSTCGSHLTVVSLFYCSVMKMYLSPTSEHEAGMQKLVTLFYSVGTPLLNPVIYSLRNKDMKNALQKILRT

SEQ ID NO: 15 - DNA, 918 bp, Mus musculus
  source 1..918: mol_type = genomic DNA
atggaactgaacaggacccagctgactgaatttgttctcagaggaataacagatcgttcagagctgcaagtccccctgttcctggtgttctttctcatctatgttatcaccatggtgggcaaccttggcttaatctttgtcatctggaaggaccctcatcttcacacacccatgtaccttttccttggaaatttggcctttgctgatgcctgtaattcatcctctgtgacaccaaagatgcttatgaaatttttaaataagaatgacatgatatccatgggtgagtgttttgctcaattttatttcttttgttcaagtgtaactgcagaagccttcattctggtagctatggcctatgaccgctatgtagccatatgcaaacctctgctctatgtagtggtgatgtccaacagactctgtattcagttcataggtgtatcctatctaattggacttctacatggcttacttcatgtaggattgttatttaggttaacgttttgtagttccaatgtaatagattatttctactgtgacatcctgccactttataggatttcttgcactgacccatcgatcaatgtactggtagctttcattatgggtattttattacaagtgagtacctttatgagtattatagtctcctatgtccgtgtcctctttgccatcctgagaacaaagtctgagaggggcagaaacaaagccttctctacttgcagttcccacctgtcatctgtgtctttgttctatggcactctcttcatcatatatgtcctctctggctctgacacagataattatcagggtaaaatgtattcactgttctataccattatcattcctctgctaaaccccttcatttacagcctaagaaataaagaagtcatcggtgccttgagaaaagtcagaaaatga

SEQ ID NO: 16 - protein, 305 aa, Mus musculus
MELNRTQLTEFVLRGITDRSELQVPLFLVFFLIYVITMVGNLGLIFVIWKDPHLHTPMYLFLGNLAFADACNSSSVTPKMLMKFLNKNDMISMGECFAQFYFFCSSVTAEAFILVAMAYDRYVAICKPLLYVVVMSNRLCIQFIGVSYLIGLLHGLLHVGLLFRLTFCSSNVIDYFYCDILPLYRISCTDPSINVLVAFIMGILLQVSTFMSIIVSYVRVLFAILRTKSERGRNKAFSTCSSHLSSVSLFYGTLFIIYVLSGSDTDNYQGKMYSLFYTIIIPLLNPFIYSLRNKEVIGALRKVRK

SEQ ID NO: 17 - DNA, 951 bp, Mus musculus
  source 1..951: mol_type = genomic DNA
atgcctggggtcaatacctccagcctgacaccaagatactttattctcaatgggattcctgggttggaagctgcacacatctggatctctctgccattcttcattatgtacctcattgctgtcacaggtaactgtggacttatctacctcatcagtcatgaggaggctctgcaccggcccatgtactactttctagccatgttgtctgctacagatatttctgggtgtaatacaattgtccccagtatgttatgcatcttttggttcagtgtcaaggagattgatttcaatgcctgccttgtacagatgtttttcatccacatgttaacaggcatggagtctggtgtgctcatgcttatggctctcgaccgctatgtggctatatgctatccattacgctatactaccatactcaccaacactatgattaccaagattggattggcagcacttgttagaagtgtgttactcatggtcccttttgctttcctgatcaagcgtcttccatactgtagaggaaacctcatccaacatacctattgtgatcacatggctgtggctaaactatcctgtggcaatattaagattaatgctatctatggtcttataattgctatatttattgggggttttgatatattctgtatctccatgtcttatgccatgattatccatgctgtggtgaagctatcttcggcagatgctcgccataaagccttcagtacctgtacatcacacatatgtgctattgttattacctatgtcccagcattcttcaacttctttactcatcgctttgggagaaccactataccccatcatatccacattattatagccaacctgtatctattgctacctcccaccttgaatccaattgtatatggagtaaagaccaagcagattcgtgaaggtgtgatcaaactgtttgctagacaaaaagttgtttga

SEQ ID NO: 18 - protein, 316 aa, Mus musculus
MPGVNTSSLTPRYFILNGIPGLEAAHIWISLPFFIMYLIAVTGNCGLIYLISHEEALHRPMYYFLAMLSATDISGCNTIVPSMLCIFWFSVKEIDFNACLVQMFFIHMLTGMESGVLMLMALDRYVAICYPLRYTTILTNTMITKIGLAALVRSVLLMVPFAFLIKRLPYCRGNLIQHTYCDHMAVAKLSCGNIKINAIYGLIIAIFIGGFDIFCISMSYAMIIHAVVKLSSADARHKAFSTCTSHICAIVITYVPAFFNFFTHRFGRTTIPHHIHIIIANLYLLLPPTLNPIVYGVKTKQIREGVIKLFARQKVV

SEQ ID NO: 19 - DNA, 954 bp, Mus musculus
  source 1..954: mol_type = genomic DNA
atgctgatttccaacaactcatatgaagccccgcagtctttcattcttaatggaattcctggtctcgaagcagtgcatatatggatctctcttccactctgtacaatgtacatcatctccctagtaggcaaccttggccttgtatatctcatttactatgaggaatccttacatcgcccaatgtatttctttctggccatgctttctctcatagacctgtttacttgcacaaccactgtccccaatgccctcttcattttctggttcaaactcaaggaaattaacttcactgcttgcctagttcagatgttctttgtgcacggattcacaggtgtggagtctggggtactcatgctcatggccttggaccgctatgtggccatttgctacccactacgctatgcaaccatacttaccaaccctgtcattgccaaagctgggcttgccaccttcttgagaggtgtgttactgatgattccttttccattcttggttaaacgtttgcccttctgccgaagcaatgtcatctcccatacatattgtgaccacatgtctgtggtaaagttatcctgtgccagcatcaaaatcaatgtcatctatggtctcatggttgcacttctgattggagtgtttgacatatgttgtatatctgtgtcctacactatgatcctccgggcagtggtcagcctgtcctctgcagatgctcggcagaaggccttcagcacctgcacagcccacatatctgccatcatcattacttatgttccagccttcttcaccttctttactcatcgttttggaggtcacaccatccctccttctcttcatatcattgtggctaatctttatcttcttctccctccaactctaaatcccattgtttatgggatgaagaccaaacagatcagagatagtatcattaaattctttcacggtgaaaaaggttcaaggtga

SEQ ID NO: 20 - protein, 317 aa, Mus musculus
MLISNNSYEAPQSFILNGIPGLEAVHIWISLPLCTMYIISLVGNLGLVYLIYYEESLHRPMYFFLAMLSLIDLFTCTTTVPNALFIFWFKLKEINFTACLVQMFFVHGFTGVESGVLMLMALDRYVAICYPLRYATILTNPVIAKAGLATFLRGVLLMIPFPFLVKRLPFCRSNVISHTYCDHMSVVKLSCASIKINVIYGLMVALLIGVFDICCISVSYTMILRAVVSLSSADARQKAFSTCTAHISAIIITYVPAFFTFFTHRFGGHTIPPSLHIIVANLYLLLPPTLNPIVYGMKTKQIRDSIIKFFHGEKGSR

SEQ ID NO: 21 - DNA, 936 bp, Mus musculus
  source 1..936: mol_type = genomic DNA
atgcaaaaccagagttttgtaacagaattcatattccttggactttcacagaaccctaaagtccagaaaatagtttttattgtatttttatttgtctacattgcaactgttgggggcaacatgataattgtggtgaccattgtctgtagcccagcattgatagactgccccatgtacttctttttggcattcttgtccctattggatgcatgcttctcttctgtcatcacaccaaagatggttgtggactccctgtatgagaagaaaactatctcctttgaaggatgtatgatgcagttatttgctgagcacttccttgcagcagtagaagtgattgtcttgacagccatggcctatgaccgctatgtagcaatttgcaagcccttgcactactcttccatcatgaactggaggctctgtggcacacttatggggatagcatggacagggggcttcttgcattctatcatacaaattatcttcacgttgcaattgcccttctgtggaccaaatgtcatcgatcatttcatgtgtgacttgttcccattactggaacttgcctgcactgatactcatatctttggccttttagtggttgccaacagtgggtctatctgcatcataatcttctctattttgctggtctcctatggtgtcatcctgttctctctgaaagctcacagttctgaagggcgatggaaagctctctccacatgtggatcccacattgcagttgtggttttgttctttgtcccgtgtatatttatttatgcacgtcctccatctgctttctcctttgataaaatggtggcgatattttatactatcctaactcccttgctcaatcctgtgatttatacttttcggaataaggacatgaaaaatgctatgaagaaagtgtggaagaggttggcagtggtttctgatggaaagtga

SEQ ID NO: 22 - protein, 311 aa, Mus musculus
MQNQSFVTEFIFLGLSQNPKVQKIVFIVFLFVYIATVGGNMIIVVTIVCSPALIDCPMYFFLAFLSLLDACFSSVITPKMVVDSLYEKKTISFEGCMMQLFAEHFLAAVEVIVLTAMAYDRYVAICKPLHYSSIMNWRLCGTLMGIAWTGGFLHSIIQIIFTLQLPFCGPNVIDHFMCDLFPLLELACTDTHIFGLLVVANSGSICIIIFSILLVSYGVILFSLKAHSSEGRWKALSTCGSHIAVVVLFFVPCIFIYARPPSAFSFDKMVAIFYTILTPLLNPVIYTFRNKDMKNAMKKVWKRLAVVSDGK

SEQ ID NO: 23 - DNA, 975 bp, Homo sapiens
  source 1..975: mol_type = genomic DNA
atgcctctatttaattcattatgctggtttccaacaattcatgtgactcctccatcttttattcttaatggaatacctggtctggaaagagtacatgtatggatctccctcccactctgcacaatgtacatcatcttccttgtggggaatcttggtcttgtgtacctcatttattatgaggagtccttacatcatccgatgtattttttttttggccatgctctctccctcattgacctccttacctgcaccaccactctacccaatgcactctgcatcttctggttcagtctcaaagaaattaacttcaatgcttgcttggcccagatgttctttgttcatgggttcacaggtgtggagtctggggtgctcatgctcatggctctagaccgctatgtagccatttgctaccctttgcgttatgctaccacactcaccaaccctatcattgccaaggctgagcttgccaccttcctgaggggtgtattgctgatgattcctttcccattcttggttaagcgtttgcctttctgccaaagcaatattatctcccatacgtactgcgaccacatgtctgtagtaaagctatcttgtgccagcatcaaggtcaatgtaatctatggtctaatggttgctctcctgattggagtgtttgacatttgttgtatatctttgtcttacactttgatcctcaaggcagcgatcagcctctcttcatcagatgctcggcagaaggctttcagcacctgcactgcccatatatctgccatcatcatcacctatgttccagcattcttcactttctttgcccaccgttttgggggacacacaattcccccttctcttcacatcattgtggctaatctttatcttcttcttcccccaactctaaaccctattgtttatggagtaaagacaaaacagatacgcaagagtgtcataaagttcttccagggtgataagggtgcaggttga

SEQ ID NO: 24 - protein, 324 aa, Homo sapiens
MPLFNSLCWFPTIHVTPPSFILNGIPGLERVHVWISLPLCTMYIIFLVGNLGLVYLIYYEESLHHPMYFFFGHALSLIDLLTCTTTLPNALCIFWFSLKEINFNACLAQMFFVHGFTGVESGVLMLMALDRYVAICYPLRYATTLTNPIIAKAELATFLRGVLLMIPFPFLVKRLPFCQSNIISHTYCDHMSVVKLSCASIKVNVIYGLMVALLIGVFDICCISLSYTLILKAAISLSSSDARQKAFSTCTAHISAIIITYVPAFFTFFAHRFGGHTIPPSLHIIVANLYLLLPPTLNPIVYGVKTKQIRKSVIKFFQGDKGAG

SEQ ID NO: 25 - DNA, 939 bp, Homo sapiens
  source 1..939: mol_type = genomic DNA
atggagaaatggaatcacacttcaaatgatttcattttgttgggtctgcttcccccaaatcaaactggaatatttctcttgtgccttatcatcctcatattctttctggcctcggtgggtaactcggccatgattcacctcatccacgtggatcctcgtctccacacaccgatgtactttcttctcagccagctctcccttatggacctgatgtacatctccaccaccgtccccaagatggcgtacaacttcctgtccggccagaaaggcatctccttcctgggatgtggtgtgcaaagcttcttcttcctgaccatggcgtgttctgaaggcttactcctgacctccatggcctacgaccgttatttggccatctgccactctctctattatcctatccgcatgagtaaaatgatgtgtgtgaagatgattggaggctcttggacactggggtccatcaactccttggcacacacagtctttgcccttcatattccctactgcaggtctagggctattgaccatttcttctgcgatgtcccagccatgttgcttcttgcctgtacagatacttgggtctatgaatatatggtttttgtaagtacaagcctctttctccttttccctttcattggcatcacttcttcctgtggccgagtcctatttgctgtctatcatatgcactcaaaggaggggagaaaaaaggccttcaccaccatttcaacacatttaactgtagtgatcttttactatgcaccttttgtctacacctatcttcggcccaggaatctccgctcaccagctgaagacaagatcctggcagtcttctacaccatccttacccccatgctcaatcccattatctacagcctgaggaataaggaagtcctgggggctatgaggagagtgtttgggatattctctttcctgaaagaataa

SEQ ID NO: 26 - protein, 312 aa, Homo sapiens
MEKWNHTSNDFILLGLLPPNQTGIFLLCLIILIFFLASVGNSAMIHLIHVDPRLHTPMYFLLSQLSLMDLMYISTTVPKMAYNFLSGQKGISFLGCGVQSFFFLTMACSEGLLLTSMAYDRYLAICHSLYYPIRMSKMMCVKMIGGSWTLGSINSLAHTVFALHIPYCRSRAIDHFFCDVPAMLLLACTDTWVYEYMVFVSTSLFLLFPFIGITSSCGRVLFAVYHMHSKEGRKKAFTTISTHLTVVIFYYAPFVYTYLRPRNLRSPAEDKILAVFYTILTPMLNPIIYSLRNKEVLGAMRRVFGIFSFLKE

SEQ ID NO: 27 - DNA, 879 bp, Homo sapiens
  source 1..879: mol_type = genomic DNA
atgagtgtaacagaaaatacgctcatgatcctcctcattcgcagtgactcccgactccacactccaatgtattttctgctcagccatctctccttaatggatatcttgcatgtttccaacatcgttcccaaaatggtcactaactttctgtcaggcagcagaactatttcatttgcaggttgtgggttccaggtatttctgtccctcaccctcctgggtggtgagtgccttctcctggctgcaatgtcctgtgatcgctatgtggctatctgtcacccgctgcgctatccgattcttatgaaggagtatgccagcgctctcatggctggaggctcctggctcattggggttttcaactccacagtccacacagcttatgcactgcagtttcccttctgtggctctagggcaattgatcacttcttctgtgaagtccctgccatgttgaagttgtcctgtgcagacacaacacgctatgaacgaggggtttgtgtaagtgctgtgatcttcctgctgatccctttctccttgatctctgcttcttatggccaaattattcttactgtcctccagatgaaatcatcagaggcaaggaaaaagtcattttccacttgttccttccacatgattgtggtcacgatgtactatgggccatttatttttacatatatgagacctaaatcataccacactccagggcaggataagttcctggcaatattctatacgatcctcacacccacactcaaccctttcatctacagctttaggaataaagatgttctggcggtgatgaaaaatatgctcaaaagtaactttctgcacaaaaaaatgaataggaaaattcctgaatgtgtgttctgtctatttctatgttaa

SEQ ID NO: 28 - protein, 292 aa, Homo sapiens
MSVTENTLMILLIRSDSRLHTPMYFLLSHLSLMDILHVSNIVPKMVTNFLSGSRTISFAGCGFQVFLSLTLLGGECLLLAAMSCDRYVAICHPLRYPILMKEYASALMAGGSWLIGVFNSTVHTAYALQFPFCGSRAIDHFFCEVPAMLKLSCADTTRYERGVCVSAVIFLLIPFSLISASYGQIILTVLQMKSSEARKKSFSTCSFHMIVVTMYYGPFIFTYMRPKSYHTPGQDKFLAIFYTILTPTLNPFIYSFRNKDVLAVMKNMLKSNFLHKKMNRKIPECVFCLFLC

SEQ ID NO: 29 - DNA, 1113 bp, Homo sapiens
  source 1..1113: mol_type = genomic DNA
atgttctcaatgacaacagaagcactcaataattttgcacttggatgtaccaacttgttaatgactatgataccacaaattgatctgaagcaaattttcctttgtcctaattgcagactatacatgatccctgttggagctttcatcttttccttgggaaacatgcaaaaccaaagctttgtaactgagtttgtcctcctgggactttcacagaatccaaatgttcaggaaatagtatttgttgtatttttgtttgtctacattgcaactgttgggggcaacatgctaattgtagtaaccattctcagcagccctgctcttctggtgtctcctatgtacttcttcttgggcttcctgtccttcctggatgcgtgcttctcatctgtcatcaccccaaagatgattgtagactccctctatgtgacaaaaaccatctcttttgaaggctgcatgatgcagctctttgctgaacacttctttgctggggtggaggtgattgtcctcacagccatggcctatgatcgttatgtggccatttgcaagcccttgcattactcttctatcatgaacaggaggctctgtggcattctgatgggggtagcctggacagggggcctcttgcattccatgatacaaattctttttactttccagcttcccttttgtggccccaatgtcatcaatcactttatgtgtgacttgtacccgttactggagcttgcctgcactgatactcacatctttggcctcatggtggtcatcaacagtgggtttatctgcatcataaacttctccttgttgcttgtctcctatgctgtcatcttgctctctctgagaacacacagttctgaagggcgctggaaagctctctccacctgtggatctcacattgctgttgtgattttgttctttgtcccatgcatatttgtatatacacgacctccatctgctttttcccttgacaaaatggcggcaatattttatatcatcttaaatcccttgctcaatcctttgatttacactttcaggaataaggaagtaaaacaggccatgaggagaatatggaacagactgatggtggtttctgatgagaaagaaaatattaaactttaa

SEQ ID NO: 30 - protein, 370 aa, Homo sapiens
MFSMTTEALNNFALGCTNLLMTMIPQIDLKQIFLCPNCRLYMIPVGAFIFSLGNMQNQSFVTEFVLLGLSQNPNVQEIVFVVFLFVYIATVGGNMLIVVTILSSPALLVSPMYFFLGFLSFLDACFSSVITPKMIVDSLYVTKTISFEGCMMQLFAEHFFAGVEVIVLTAMAYDRYVAICKPLHYSSIMNRRLCGILMGVAWTGGLLHSMIQILFTFQLPFCGPNVINHFMCDLYPLLELACTDTHIFGLMVVINSGFICIINFSLLLVSYAVILLSLRTHSSEGRWKALSTCGSHIAVVILFFVPCIFVYTRPPSAFSLDKMAAIFYIILNPLLNPLIYTFRNKEVKQAMRRIWNRLMVVSDEKENIKL

SEQ ID NO: 31 - DNA, 930 bp, Homo sapiens
  source 1..930: mol_type = genomic DNA
atggatatatcagagggaaataagactcttgtgacagagtttgttctcacaggacttacagatcgaccatggctgcacgtcctcttctttgttgtgtttttggtggtctatctcatcaccatggtgggcaaccttggactgatagttctaatttggaacgacccccatcttcatatgcccatgtacttattccttggtggtttagccttttcagatgcttgtacttcaacctctataacccctaggatgctggtcaatttcttagacaagactgcaatgatatccctagctgagtgcatcacccagttttacttttttgcttccagtgcaactacagaatgcttcctcctggtgatgatggcctatgaccgctatgtagccatatgtaatcccttgctttatccagtgatgatgtccaacaaactcagcgctcagttgctaagtatttcatatgtaattggtttcctgcatcctctggttcatgtgagtttactattgcgactaactttctgcaggtttaacataatacattatttctactgtgaaattttacaactgttcaaaatttcatgcaatggtccatctattaacgcactaatgatatttatttttggtgcttttatacaaatacccactttaatgactatcataatctcttatactcgtgtgctctttgatattctgaaaaaaaagtctgaaaagggcagaagcaaagccttctccacatgcggcgcccatctgctttctgtctcattgtactacggaactctgatcttcatgtatgtgcgtcctgcatctggcttagctgaagaccaagacaaagtgtattctctgttttacacgattataattcccctgctaaacccatttatttacagcttgagaaataaaaaagtcatgcatgcattgagaagagttataaggaagtaa

SEQ ID NO: 32 - protein, 309 aa, Homo sapiens
MDISEGNKTLVTEFVLTGLTDRPWLHVLFFVVFLVVYLITMVGNLGLIVLIWNDPHLHMPMYLFLGGLAFSDACTSTSITPRMLVNFLDKTAMISLAECITQFYFFASSATTECFLLVMMAYDRYVAICNPLLYPVMMSNKLSAQLLSISYVIGFLHPLVHVSLLLRLTFCRFNIIHYFYCEILQLFKISCNGPSINALMIFIFGAFIQIPTLMTIIISYTRVLFDILKKKSEKGRSKAFSTCGAHLLSVSLYYGTLIFMYVRPASGLAEDQDKVYSLFYTIIIPLLNPFIYSLRNKKVMHALRRVIRK

SEQ ID NO: 33 - DNA, 939 bp, Homo sapiens
  source 1..939: mol_type = genomic DNA
atgatgggtagaaggaatgacacaaatgtggctgacttcatccttacgggactgtcagactctgaagaggtccagatggctctgtttatgctatttctcctcatatacctaattactatgctggggaatgtggggatgctattgataatccgcctggacctccagcttcacactcccatgtattttttccttactcacctgtcatttattgacctcagttactcaactgtcgtcacacctaaaaccttagcgaacttactgacttccaactatatttccttcacgggctgctttgcccagatgttctgttttgtcttcttgggtactgctgaatgttatcttctctcctcaatggcctatgatcgctatgcagcgatctgcagtcctctacactacacagttattatgcccaaaaggctctgcctcgctctcatcactgggccttatgtgattggctttatggactcctttgtcaatgtggtttccatgagcagattgcatttctgtgactcaaacataattcatcactttttctgtgacacttccccaattttagctctgtcctgcactgacacagacaacactgaaatgctgatattcattatcgctggttccaccctgatggtgtcccttatcacaatatctgcatcctatgtgtccattctctctaccatcctgaaaattaattccacttcaggaaagcagaaagctttctctacttgcgtctctcatctcttgggagtcaccatcttctatggaactatgatttttacttacttaaagccaagaaagtcttattccttgggaagagatcaagtggctcctgtgttttatactattgtgattcccatgctgaatccactcatttatagtcttagaaacagagaagtgaaaaatgctctcattagagtcatgcagagaagacaggactccaggtag

SEQ ID NO: 34 - protein, 312 aa, Homo sapiens
MMGRRNDTNVADFILTGLSDSEEVQMALFMLFLLIYLITMLGNVGMLLIIRLDLQLHTPMYFFLTHLSFIDLSYSTVVTPKTLANLLTSNYISFTGCFAQMFCFVFLGTAECYLLSSMAYDRYAAICSPLHYTVIMPKRLCLALITGPYVIGFMDSFVNVVSMSRLHFCDSNIIHHFFCDTSPILALSCTDTDNTEMLIFIIAGSTLMVSLITISASYVSILSTILKINSTSGKQKAFSTCVSHLLGVTIFYGTMIFTYLKPRKSYSLGRDQVAPVFYTIVIPMLNPLIYSLRNREVKNALIRVMQRRQDSR

SEQ ID NO: 35 - DNA, 936 bp, Homo sapiens
  source 1..936: mol_type = genomic DNA
atgaaaatcttcaacagccccagcaactccagcaccttcactggcttcatcctcctgggcttcccttgccccagggaggggcagatcctcctctttgtgctcttcactgttgtttacctcctgaccctcatgggcaatggttccatcatctgtgctgtgcactgggatcagagactccacgcccccatgtacatcctgctcgccaacttctccttcttggagatatgttatgtcacctccacagtccccagcatgctggccaacttcctctctgacaccaagatcatctcgttctctggctgcttcctccagttctactttttcttctccttgggctctacagaatgctttttcctggcagttatggcatttgatcgataccttgccatctgtcggcctctacgctatccaaccattatgaccagacgtctctgtaccaatcttgtggtcaattgctgggtacttggtttcatctggttcttgattcctatcgtcaacatctcccaaatgtccttctgtggatctaggattattgaccacttcctatgtgacccagctcctcttctaactctcacttgcaaaaaaggccctgtgatagagcttgtcttttctgtcttaagtcctctgcctgtctttatgctctttctcttcattgtggggtcctatgctctggtcgtgagagctgtgttgagggtcccttcagcagctgggagaagaaaggctttctccacctgtgggtctcacctggctgtggtttcactgttctacggctcagtactggtcatgtatgggagcccaccatctaagaatgaagctggaaagcagaagactgtgactctgttttattctgttgttaccccactgcttaaccctgtgatatatagtcttaggaacaaagatatgagaaaagctctgaagaaattttggggaacataa

SEQ ID NO: 36 - protein, 311 aa, Homo sapiens
MKIFNSPSNSSTFTGFILLGFPCPREGQILLFVLFTVVYLLTLMGNGSIICAVHWDQRLHAPMYILLANFSFLEICYVTSTVPSMLANFLSDTKIISFSGCFLQFYFFFSLGSTECFFLAVMAFDRYLAICRPLRYPTIMTRRLCTNLVVNCWVLGFIWFLIPIVNISQMSFCGSRIIDHFLCDPAPLLTLTCKKGPVIELVFSVLSPLPVFMLFLFIVGSYALVVRAVLRVPSAAGRRKAFSTCGSHLAVVSLFYGSVLVMYGSPPSKNEAGKQKTVTLFYSVVTPLLNPVIYSLRNKDMRKALKKFWGT

SEQ ID NO: 37 - DNA, 966 bp, Homo sapiens
  source 1..966: mol_type = genomic DNA
atgtctggggacaacagctccagcctgaccccaggattctttatcttgaatggcgttcctgggctggaagccacacacatctggatctccctgccattctgctttatgtacatcattgctgtcgtggggaactgtgggctcatctgcctcatcagccatgaggaggccctgcaccggcccatgtactacttcctggccctgctctccttcactgatgtcaccttgtgcaccaccatggtacctaatatgctgtgcatattctggttcaacctcaaggagattgactttaacgcctgcctggcccagatgttttttgtccatatgctgacagggatggagtctggggtgctcatgctcatggccctggaccgctatgtggccatctgctaccccttacgctatgccaccatccttaccaaccctgtcatcgccaaggctggtcttgccaccttcttgaggaatgtgatgctcatcatcccattcactctcctcaccaagcgcctgccctattgccgggggaacttcatcccccacacctactgtgaccatatgtctgtggccaaggtatcctgtggcaatttcaaggtcaatgctatttatggtctgatggttgctctcctgattggtgtgtttgatatctgctgtatctctgtatcttacactatgattttgcaggctgttatgagcctgtcatcagcagatgctcgtcacaaagccttcagcacctgcacatctcacatgtgttccattgtgatcacctatgttgctgcttttttcacttttttcactcatcgttttgtaggacacaatatcccaaaccacatacacatcatcgtggccaacctttatctgctactgcctcctaccatgaacccaattgtttatggagtcaagaccaagcagattcaggaaggtgtaattaaatttttacttggagacaaggttagttttacctatgacaaatga

SEQ ID NO: 38 - protein, 321 aa, Homo sapiens
MSGDNSSSLTPGFFILNGVPGLEATHIWISLPFCFMYIIAVVGNCGLICLISHEEALHRPMYYFLALLSFTDVTLCTTMVPNMLCIFWFNLKEIDFNACLAQMFFVHMLTGMESGVLMLMALDRYVAICYPLRYATILTNPVIAKAGLATFLRNVMLIIPFTLLTKRLPYCRGNFIPHTYCDHMSVAKVSCGNFKVNAIYGLMVALLIGVFDICCISVSYTMILQAVMSLSSADARHKAFSTCTSHMCSIVITYVAAFFTFFTHRFVGHNIPNHIHIIVANLYLLLPPTMNPIVYGVKTKQIQEGVIKFLLGDKVSFTYDK

SEQ ID NO: 39 - DNA, 915 bp, Homo sapiens
  source 1..915: mol_type = genomic DNA
atgtttatattaataagcttcacagaagaatttgatgtgcaagtcttcctatttttattatttttagcaatctatctattcactctaataggcaatttagggctggttgtaccgatcattggggatttctggcttcacagcccaatgtactattttcttggtgttttatcattcttggatgtctgctattctacagttgtcactccaaaaatgttggtcaatttcctggcaaaaaataaatctatttcatttcttggatgtgcaacacagatgtttcttgcttgtacttttggaaccacagaatgctttctcttggctgcaatggcttatgatcgctatgtagccatctacaaccctctcctgtattcagtgagcatgtcacccagagtctatgtgccactcatcactgcttcctatgttgctagcattttacatgctactatacatacagtggctacatttagcctgtccttctgtggatccaatgaaattaggcatgtcttttgtaatatgcctcctctccttgctatttcttgttctgacactcacgtaatccagcttctattcttctactttgtgggctctattgagatagtcactatcctgattgtcctgatctcctatggttttattctgttggccattctgaagatgcagtctgctgaagggaggagaaaagtcttctctacatgtggagctcacctaactggagtgacaatttatcatgggacaatcctcttcatgtatgtgagaccaagttccagctacacttcggacaatgacatgatagtgtcaatattttataccattgtgattcccatgctgaatcccatcatctacagtttgcggaacaaagatgtaaaggaggcaatcaaaagattgcttgtgagaaattggttcataaataagttatag

SEQ ID NO: 40 - protein, 304 aa, Homo sapiens
MFILISFTEEFDVQVFLFLLFLAIYLFTLIGNLGLVVPIIGDFWLHSPMYYFLGVLSFLDVCYSTVVTPKMLVNFLAKNKSISFLGCATQMFLACTFGTTECFLLAAMAYDRYVAIYNPLLYSVSMSPRVYVPLITASYVASILHATIHTVATFSLSFCGSNEIRHVFCNMPPLLAISCSDTHVIQLLFFYFVGSIEIVTILIVLISYGFILLAILKMQSAEGRRKVFSTCGAHLTGVTIYHGTILFMYVRPSSSYTSDNDMIVSIFYTIVIPMLNPIIYSLRNKDVKEAIKRLLVRNWFINKL

SEQ ID NO: 41 - DNA, 936 bp, Homo sapiens
  source 1..936: mol_type = genomic DNA
atggaaaaaataaacaacgtaactgaattcattttctggggtctttctcagagcccagagattgagaaagtttgttttgtggtgttttctttcttctacataatcattcttctgggaaatctcctcatcatgctgacagtttgcctgagcaacctgtttaagtcacccatgtatttctttctcagcttcttgtcttttgtggacatttgttactcttcagtcacagctcccaagatgattgttgacctgttagcaaaggacaaaaccatctcctatgtggggtgcatgttgcaactgtttggagtacatttctttggttgcactgagatcttcatccttactgtaatggcctatgatcgttatgtggctatctgtaaacccctacattatatgaccatcatgaaccgggagacatgcaataaaatgttattagggacgtgggtaggtgggttcttacactccattatccaagtggctctggtagtccaactacccttttgtggacccaatgagatagatcactacttttgtgatgttcaccctgtgttgaaacttgcctgcacagaaacatacattgttggtgttgttgtgacagccaacagtggtaccattgctctggggagttttgttatcttgctaatctcctacagcatcatcctagtttccctgagaaagcagtcagcagaaggcaggcgcaaagccctctccacctgtggctcccacattgccatggtcgttatctttttcggcccctgtacttttatgtacatgcgccctgatacgaccttttcagaggataagatggtggctgtattttacaccattatcactcccatgttaaatcctctgatttatacactgagaaatgcagaagtaaagaatgcaatgaagaaactgtggggcagaaatgttttcttggaggctaaagggaaatag

SEQ ID NO: 42 - protein, 311 aa, Homo sapiens
MEKINNVTEFIFWGLSQSPEIEKVCFVVFSFFYIIILLGNLLIMLTVCLSNLFKSPMYFFLSFLSFVDICYSSVTAPKMIVDLLAKDKTISYVGCMLQLFGVHFFGCTEIFILTVMAYDRYVAICKPLHYMTIMNRETCNKMLLGTWVGGFLHSIIQVALVVQLPFCGPNEIDHYFCDVHPVLKLACTETYIVGVVVTANSGTIALGSFVILLISYSIILVSLRKQSAEGRRKALSTCGSHIAMVVIFFGPCTFMYMRPDTTFSEDKMVAVFYTIITPMLNPLIYTLRNAEVKNAMKKLWGRNVFLEAKGK

SEQ ID NO: 43 - DNA, 24 bp, synthetic construct
  misc_feature 1..24: note = FLAG tag
  source 1..24: mol_type = other DNA
gattacaaggacgacgacgataag

SEQ ID NO: 44 - protein, 8 aa, synthetic construct
  REGION 1..8: note = FLAG tag
DYKDDDDK

SEQ ID NO: 45 - DNA, 60 bp, synthetic construct
  misc_feature 1..60: note = Rho tag
  source 1..60: mol_type = other DNA
atgaacgggaccgagggcccaaacttctacgtgcctttctccaacaagacgggcgtggtg

SEQ ID NO: 46 - protein, 20 aa, synthetic construct
  REGION 1..20: note = Rho tag
MNGTEGPNFYVPFSNKTGVV

SEQ ID NO: 47 - DNA, 51 bp, synthetic construct
  misc_feature 1..51: note = Lucy tag
  source 1..51: mol_type = other DNA
atgagaccccagatcctgctgctcctggccctgctgaccctaggcctggct

SEQ ID NO: 48 - protein, 17 aa, synthetic construct
  REGION 1..17: note = Lucy tag
MRPQILLLLALLTLGLA

SEQ ID NO: 49 - DNA, 1376 bp, Homo sapiens
  source 1..1376: mol_type = genomic DNA
atgggtctgtgctacagtctgcggccgctgcttttcgggggcccaggggacgacccctgcgcggcctcggagccgccggtggaggacgcgcagcccgccccggccccggccctggccccagtccgggcggccgcaagggacacggcccggaccctgctccctcggggcggcgaagggagcccggcatgcgctcggcccaaagcagacaagccgaaggagaagcggcagcgcaccgagcagcttagtgccgaggagcgcgaggcggccaaggagcgcgaggcggtcaaggaggcgaggaaagtgagccggggcatcgaccgcatgctgcgcgaccagaagcgcgacctgcagcagacgcaccggctcctgctgctcggggctggtgagtctgggaaaagcactatcgtcaaacagatgaggatcctgcacgtcaatgggtttaatcccgaggaaaagaaacagaaaattctggacatccggaaaaatgttaaagatgctatcgtgacaattgtttcagcaatgagtactataatacctccagttccgctggccaaccctgaaaaccaatttcgatcagactacatcaagagcatagcccctatcactgactttgaatattcccaggaattctttgaccatgtgaaaaaactttgggacgatgaaggcgtgaaggcatgctttgagagatccaacgaataccagctgattgactgtgcacaatacttcctggaaagaatcgacagcgtcagcttggttgactacacacccacagaccaggacctcctcagatgcagagttctgacatctgggatttttgagaacgattccaagtggacaaagtaaacttccacatgtttgatgttggtggccagagggatgagaggagaaaatggatccagtgctttaacgatgtcacagctatcatttacgtcgcagcctgcagtagctacaacatggtgattcgagaagataacaacaccaacaggctgagagagtccctggatctttttgaaagcatctggaacaacaggtggttacggaccatttctatcatcttgttcttgaacaaacaagatatgctggcagaaaaagtcttggcagggaaatcaaaaattgaagactatttcccagaatatgcaaattatactgttcctgaagacgcaacaccagatgcaggagaagatcccaaagttacaagagccaagttctttatccgggacctgtttttgaggatcagcacggccaccggtgacggcaaacattactgctacccgcacttcacctgcgccgtggacacagagaacatccgcagggtgttcaacgactgccgcgacatcatccagcggatgcacctcaagcagtatgagctcttgtga

SEQ ID NO: 50 - protein, 458 aa, Homo sapiens
MGLCYSLRPLLFGGPGDDPCAASEPPVEDAQPAPAPALAPVRAAARDTARTLLPRGGEGSPACARPKADKPKEKRQRTEQLSAEEREAAKEREAVKEARKVSRGIDRMLRDQKRDLQQTHRLLLLGAGESGKSTIVKQMRILHVNGFNPEEKKQKILDIRKNVKDAIVTIVSAMSTIIPPVPLANPENQFRSDYIKSIAPITDFEYSQEFFDHVKKLWDDEGVKACFERSNEYQLIDCAQYFLERIDSVSLVDYTPTDQDLLRCRVLTSGIFETRFQVDKVNFHMFDVGGQRDERRKWIQCFNDVTAIIYVAACSSYNMVIREDNNTNRLRESLDLFESIWNNRWLRTISIILFLNKQDMLAEKVLAGKSKIEDYFPEYANYTVPEDATPDAGEDPKVTRAKFFIRDLFLRISTATGDGKHYCYPHFTCAVDTENIRRVFNDCRDIIQRMHLKQYELL

SEQ ID NO: 51 - DNA, 1125 bp, Homo sapiens
  source 1..1125: mol_type = genomic DNA
atggcccggtcgctgacctggcgctgctgcccctggtgcctgacggaggatgagaaggccgccgcccgggtggaccaggagatcaacaggatcctcttggagcagaagaagcaggaccgcggggagctgaagctgctgcttttgggcccaggcgagagcgggaagagcaccttcatcaagcagatgcggatcatccacggcgccggctactcggaggaggagcgcaagggcttccggcccctggtctaccagaacatcttcgtgtccatgcgggccatgatcgaggccatggagcggctgcagattccattcagcaggcccgagagcaagcaccacgccagcctggtcatgagccaggacccctataaagtgaccacgtttgagaagcgctacgctgcggccatgcagtggctgtggagggatgccggcatccgggcctgctatgagcgtcggcgggaattccacctgctcgattcagccgtgtactacctgtcccacctggagcgcatcaccgaggagggctacgtccccacagctcaggacgtgctccgcagccgcatgcccaccactggcatcaacgagtactgcttctccgtgcagaaaaccaacctgcggatcgtggacgtcgggggccagaagtcagagcgtaagaaatggatccattgtttcgagaacgtgatcgccctcatctacctggcctcactgagtgaatacgaccagtgcctggaggagaacaaccaggagaaccgcatgaaggagagcctcgcattgtttgggactatcctggaactaccctggttcaaaagcacatccgtcatcctctttctcaacaaaaccgacatcctggaggagaaaatccccacctcccacctggctacctatttccccagtttccagggccctaagcaggatgctgaggcagccaagaggttcatcctggacatgtacacgaggatgtacaccgggtgcgtggacggccccgagggcagcaagaagggcgcacgatcccgacgcctcttcagccactacacatgtgccacagacacacagaacatccgcaaggtcttcaaggacgtgcgggactcggtgctcgcccgctacctggacgagatcaacctgctgtga

SEQ ID NO: 52 - protein, 374 aa, Homo sapiens
MARSLTWRCCPWCLTEDEKAAARVDQEINRILLEQKKQDRGELKLLLLGPGESGKSTFIKQMRIIHGAGYSEEERKGFRPLVYQNIFVSMRAMIEAMERLQIPFSRPESKHHASLVMSQDPYKVTTFEKRYAAAMQWLWRDAGIRACYERRREFHLLDSAVYYLSHLERITEEGYVPTAQDVLRSRMPTTGINEYCFSVQKTNLRIVDVGGQKSERKKWIHCFENVIALIYLASLSEYDQCLEENNQENRMKESLALFGTILELPWFKSTSVILFLNKTDILEEKIPTSHLATYFPSFQGPKQDAEAAKRFILDMYTRMYTGCVDGPEGSKKGARSRRLFSHYTCATDTQNIRKVFKDVRDSVLARYLDEINLL

SEQ ID NO: 53 - protein, 10 aa, synthetic construct
  REGION 1..10: note = Protein motif
MAYDRYVAIC

SEQ ID NO: 54 - protein, 7 aa, synthetic construct
  REGION 1..7: note = Protein motif
FSTCSSH

SEQ ID NO: 55 - protein, 8 aa, synthetic construct
  REGION 1..8: note = Protein motif
PMLNPFIY